SEQUENCE LISTING (4 sequences)

SEQ ID NO: 1 - protein, 248 aa, Bacillus subtilis
MTGMVTERRSVHFIAEALTENCREIFERRRHVLVGISPFNSRFSEDYIYRLIGWAKAQFKSVSVLLAGHEAANLLEALGTPRGKAERKVRKEVSRNRRFAERALVAHGGDPKAIHTFSDFIDNKAYQLLRQEVEHAFFEQPHFRHACLDMSREAIIGRARGVSLMMEEVSEDMLNLAVEYVIAELPFFIGAPDILEVEETLLAYHRPWKLGEKISNHEFSICMRPNQGYLIVQEMAQMLSEKRITSEG

SEQ ID NO: 2 - protein, 249 aa, Bacillus licheniformis
MTELIMESKHQLFKTETLTQNCNEILKRRRHVLVGISPFNSRFSEDYIHRLIAWAVREFQSVSVLLAGKEAANLLEALGTPHGKAERKVRKEVSRNRRFAEKALEAHGGNPEDIHTFSDFANQTAYRNLRMEVEAAFFDQTHFRNACLEMSHAAILGRARGTRMDVVEVSADMLELAVEYVIAELPFFIAAPDILGVEETLLAYHRPWKLGEQISRNEFAVKMRPNQGYLMVSEADERVESKSMQEERV

SEQ ID NO: 3 - protein, 405 aa, Bacillus subtilis
MSQSIKLFSVLSDQFQNNPYAYFSQLREEDPVHYEESIDSYFISRYHDVRYILQHPDIFTTKSLVERAEPVMRGPVLAQMHGKEHSAKRRIVVRSFIGDALDHLSPLIKQNAENLLAPYLERGKSDLVNDFGKTFAVCVTMDMLGLDKRDHEKISEWHSGVADFITSISQSPEARAHSLWCSEQLSQYLMPVIKERRVNPGSDLISILCTSEYEGMALSDKDILALILNVLLAATEPADKTLALMIYHLLNNPEQMNDVLADRSLVPRAIAETLRYKPPVQLIPRQLSQDTVVGGMEIKKDTIVFCMIGAANRDPEAFEQPDVFNIHREDLGIKSAFSGAARHLAFGSGIHNCVGAAFAKNEIEIVANIVLDKMRNIRLEEDFCYAESGLYTRGPVSLLVAFDGA

SEQ ID NO: 4 - protein, 406 aa, Bacillus licheniformis
MNQSLKTFSVLSEQYHENPYQYFSYLRESDPVHYEESLDSYFISRYQDVRRVLQNQDVFTTKSLAKRAEPVMRGPVLAQMKGKEHTAKRRIVLRRFIGESLDHLTPLIKENAQRLLAPHVEKGRIDLVNDFGKTFAVCVTMDILGLDKNDHQTVRNWHSGVADFITSLNQAPEDREHSLKCSEQLAEYLNPIIEERRKNPGHDLISILCTSEYEGVAMSDRDIRALILNILLAATEPADKTLALMIYHLLHHPDQMNDVLEDRTLLPQAIAETLRYKPPVQLIPRQLSQDAEIGGVELKEGTTVFCMIGAANRDPEAFEDPDKFNIHRSDLEVKSAFSGAARHLAFGSGVHNCVGAGFAKTEIELVANIVLDQLKNIRLEEDFIYRETGLYTRGPVSLNIRFDAKH

(19) United States
(12) Patent Application Publication
(10) Pub. No.: US 2025/0154515 A1
(43) Pub. Date: May 15, 2025

(54) METHOD OF GENERATING PULCHERRIMIN AND PULCHERRIMINIC ACID AND USES THEREOF

(71) Applicants: The State of Israel, Ministry of Agriculture & Rural Development, Agricultural Research Organization, Rishon-LeZion (IL); Northeastern University, Boston, MA (US)

(72) Inventors: Moshe SHEMESH, ModiIn (IL); Leticia Mara LIMA ANGELINI, Boston, MA (US); Yunrong CHAI, Chestnut Hill, MA (US)

(73) Assignees: The State of Israel, Ministry of Agriculture & Rural Development, Agricultural Research Organization, Rishon-LeZion (IL); Northeastern University, Boston, MA (US)

(21) Appl. No.: 18/945,799

(22) Filed: Nov. 13, 2024

Related U.S. Application Data
(60) Provisional application No. 63/598,190, filed on Nov. 13, 2023.

(51) Int. Cl. C12N 15/75; C07K 5/12; C12N 9/02; C12N 9/10

(52) U.S. Cl. CPC C12N 15/75; C07K 5/12; C12N 9/0071; C12N 9/104; C12Y 114/14001

(57) ABSTRACT

A composition of matter comprising genetically modified bacteria of the Bacillus genus which over-express cyclodipeptide synthase (YvmC) and/or cytochrome P450 oxidase (CypX) is disclosed. The bacteria synthesize at least twice the amount of pulcherriminic acid as compared to the amount synthesized in a non-genetically modified bacteria of a corresponding strain.

### RELATED APPLICATION(S)

This application claims the benefit of priority under 35 USC § 119 (e) of U.S. Provisional Patent Application No. 63/598,190 filed on Nov. 13, 2023, the contents of which are incorporated by reference as if fully set forth herein in their entirety.

This invention was made with government support under 1651732 awarded by the National Science Foundation. The government has certain rights in the invention.

### SEQUENCE LISTING STATEMENT

The XML file, entitled 102096SequenceListing.xml, created on Nov. 13, 2024, comprising 5,687 bytes, submitted concurrently with the filing of this application is incorporated herein by reference.

### FIELD AND BACKGROUND OF THE INVENTION

The present invention, in some embodiments thereof, relates to methods of generating pulcherrimin and pulcherriminic acid and uses thereof.

The cyclic dipeptide pulcherriminic acid produced by particular bacteria (including B. subtilis) is a non-ribosomal peptide (NRP) and a secondary metabolite. The biosynthesis pathway of pulcherriminic acid involves the activity of two key enzymes; a cyclodipeptide synthase YvmC, which catalyzes the formation of cyclic-di-leucine from two leucine-charged tRNA molecules, and a cytochrome P450 oxidase CypX, which oxidizes the cyclic dipeptide, yielding pulcherriminic acid. Once this molecule is secreted to the extracellular environment, it chelates ferric iron (Fe⁺³), turning itself into the reddish pigment pulcherrimin. The biosynthetic operon of pulcherrimin, yvmC-cypX, is regulated by AbrB, a transition-state transcriptional regulator known to repress the synthesis of secondary metabolites during the exponential phase. In addition, PchR is a negative regulator of pulcherrimin biosynthesis. This MarR-like transcription repressor directly binds to the promoter of the yvmC-cypX operon and represses expression of these biosynthetic genes.

The biological importance of both pulcherriminic acid and pulcherrimin is still not well understood. One previous study in the yeast Kluyveromyces lactis demonstrated that pulcherrimin functions as a siderophore that is taken up by the cells through a dedicated transporter. Another study in B. subtilis showed that secretion of pulcherrimin can cause growth arrest and inhibit the expansion of colony biofilms through localized iron depletion.

Background art includes Rajasekharan S K. et al., Microbial biotechnology. 2021; 14:1839-46; Amoah Y S, Nutrients. 2021; 13:4228; International Patent Application No. WO2022/018724; Shemesh and Chai, J Bacteriol. 2013 June; 195 (12): 2747-2754. Borriss, R. 2011; Arnaouteli, S. et al., Proc Natl Acad Sci USA 116, 13553-13562 (2019); Angelini et al., Biofilms and Microbiomes (2023) 9:50; doi (dot) org/10.1038/s41522-023-00418-z; Pawlikowska et al., Fermentation 2020, 6, 114; doi: 10.3390/fermentation6040114; Kregiel et al., Molecules 2023, 28 (13), 5064; doi (dot) org/10.3390/molecules28135064; Lamoureux et al DOI:doi (dot) org/10.21203/rs.3.rs-2023345/v1.

### SUMMARY OF THE INVENTION

According to an aspect of the present invention there is provided a composition of matter comprising genetically modified bacteria of the Bacillus genus which over-express cyclodipeptide synthase (YvmC) and/or cytochrome P450 oxidase (CypX), wherein the bacteria synthesize at least twice the amount of pulcherriminic acid as compared to the amount synthesized in a non-genetically modified bacteria of a corresponding strain.

According to embodiments of the invention, the genetically modified bacteria of the Bacillus genus comprise B. subtilis and/or B. licheniformis.

According to embodiments of the invention, the composition of matter is essentially devoid of biofilm.

According to embodiments of the invention, the genetically modified bacteria have a mutation in the gene srfAA.

According to embodiments of the invention, the composition further comprises iron.

According to embodiments of the invention, an amount of the iron is between 0.05-0.2 mM.

According to embodiments of the invention, the composition is devoid of iron.

According to embodiments of the invention, the amount of iron is such that the ratio of pulcherriminic acid:pulcherrimin is greater than 1:1.

According to embodiments of the invention, the composition of matter further comprises a probiotic bacteria that is not capable of endogenously synthesizing pulcherriminic acid.

According to embodiments of the invention, the composition is a cell culture which comprises a culture medium.

According to an aspect of the present invention there is provided a conditioned medium of the cell culture described herein.

According to embodiments of the invention, the culture medium comprises starch fibers of a legume of a leguminous plant.

According to embodiments of the invention, the legume comprises chickpeas.

According to embodiments of the invention, the composition of matter is lyophilized.

According to an aspect of the present invention there is provided a method of generating pulcherriminic acid comprising culturing recombinant bacteria of the Bacillus genus genetically modified to over-express cyclodipeptide synthase (YvmC) and/or cytochrome P450 oxidase (CypX) in a culture medium under conditions that the bacteria synthesize at least twice the amount of pulcherriminic acid as compared to the amount synthesized in a non-genetically modified bacteria of a corresponding strain, thereby generating pulcherriminic acid.

According to embodiments of the invention, the conditions do not promote formation of a biofilm.

According to embodiments of the invention, the culture medium is devoid of iron.

According to embodiments of the invention, the culture medium comprises iron.

According to embodiments of the invention, the culture medium comprises starch fibers of a legume of a leguminous plant.

According to embodiments of the invention, the legume comprises chickpeas.

According to embodiments of the invention, the method further comprises purifying the pulcherriminic acid from the culture medium following the culturing.

According to embodiments of the invention, the purifying is effected by size exclusion.

According to embodiments of the invention, the purifying is effected using reverse phase column chromatography.

According to embodiments of the invention, the method further comprises generating the genetically modified bacteria prior to the culturing.

According to an aspect of the present invention there is provided a composition comprising:
(i) a probiotic bacteria; and
(ii) pulcherriminic acid and/or pulcherrimin,
wherein the probiotic bacteria in the composition is incapable of endogenously synthesizing pulcherriminic acid.

According to embodiments of the invention, the composition comprises:
(i) the probiotic bacteria; and
(ii) the pulcherriminic acid.

According to embodiments of the invention, the composition is essentially devoid of biofilm.

According to embodiments of the invention, the probiotic bacteria are therapeutic probiotic bacteria.

According to an aspect of the present invention there is provided a method of enhancing a beneficial effect of a probiotic bacteria at a desired site or location comprising:
(a) contacting the site or location with the probiotic bacteria; and
(b) contacting the site or location with a preparation of purified pulcherriminic acid and/or pulcherrimin, thereby enhancing the desired effect of the probiotic bacteria.

According to embodiments of the invention, the probiotic bacteria are incapable of endogenously synthesizing pulcherriminic acid and/or pulcherrimin.

According to embodiments of the invention, the beneficial effect is a therapeutic effect.

According to embodiments of the invention, the beneficial effect is a plant-promoting effect.

According to embodiments of the invention, the contacting the location comprises administering to a subject in need thereof a therapeutically effective amount of the probiotic bacteria.

According to embodiments of the invention, the probiotic bacteria are comprised in spores.

According to embodiments of the invention, the amount of pulcherriminic acid and/or pulcherrimin is an amount which promotes the probiotic bacteria to enter a sporulation cycle.

According to embodiments of the invention, the amount of pulcherriminic acid and/or pulcherrimin is an amount which has an anti-oxidative effect on the probiotic bacteria.

According to an aspect of the present invention there is provided method of treating a yeast infection in a subject in need thereof comprising administering to the subject a therapeutically effective amount of a purified preparation of pulcherriminic acid, thereby treating the yeast infection.

According to embodiments of the invention, the yeast infection is a Candida Albicans infection.

According to embodiments of the invention, the yeast is comprised in a biofilm.

According to an aspect of the present invention there is provided method of breaking down a biofilm of yeast comprising contacting the biofilm with an effective amount of a purified preparation of pulcherriminic acid or the composition described herein, thereby breaking down the biofilm of yeast.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be 10 necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D. Strong production of pulcherrimin and activities of its biosynthetic genes during B. subtilis biofilm development. (A) Colony biofilms of the B. subtilis wild-type strain (3610, 1), the complementation strain (LA33, 2), and the pulcherrimin biosynthetic mutant ΔyvmC-cypX (YC800, 3) grown on biofilm-inducing media LBGM supplemented with 0.2 mM FeCl₃. Plates were incubated at 30° C. and images were taken every 24 h over the course of 4 days. Scale bar, 10 mm. (B) Induction of the pulcherrimin biosynthesis operon during biofilm formation using the reporter strain bearing the transcriptional fusion P_{yvmC}-gfp (LA20). Pellicle biofilms developed in LBGM and incubated statically at 30° C. Pellicles were then harvested and cells examined under fluorescence microscopy every 24 hours over the course of 4 days. Representative phase and fluorescence images from each time point were shown. Scale bar, 10 μm. (C) Violin plots demonstrating the distribution of fluorescence expressed from wild-type cells bearing the PyvmC-gfp reporter (LA20). Cells were collected at 4 different time points during pellicle biofilm development (from 24 h to 96 h, as shown in b above). Each dot represents a single cell. Fluorescence pixel quantification of cells was carried out by using the MicrobeJ. Median values are represented by dashed horizontal lines. Solid lines represent standard deviation. (D) Activities of the B. subtilis strain bearing the transcriptional reporter P_{yvmC}-lacZ (LA11). Pellicle biofilms of the reporter strain similarly developed and were collected, and β-galactosidase assays were performed. Average activities are representative of three biological replicates. Error bars represent standard deviation. M.U., Miller units.

FIGS. 2A-E. Addition of iron to growth media increases the yield of pulcherrimin, but not the activity of its biosynthetic genes. (A) Comparison of B. subtilis wild-type cell suspensions grown overnight under shaking in regular LBGM broth and in LBGM supplemented with 0.05 or 0.2 mM FeCl₃. As the concentration of FeCl₃ increases, so does the amount of reddish pigment. (B) Pellets obtained after harvesting cell suspensions of the wild type (left) and the pulcherrimin mutant (right) grown overnight in LBGM broth without or with the addition of 0.2 mM FeCl₃. Pellets contained a mix of cells and pulcherrimin, which co-precipitate after centrifugation. (C) Results of indirect pulcherrimin quantification from cell pellets obtained after centrifugation of wild-type overnight cell suspensions from (A). There is a significant increase in pulcherrimin production in samples supplemented with FeCl₃ to a final concentration of 0.05 mM compared with the ones without FeCl₃ supplementation (p=0.0151, *); significantly higher pulcherrimin production was also observed when comparing samples supplemented with 0.2 mM FeCl₃ to the ones without FeCl₃ supplementation (p=0.0002, ***). Averages are representative of three biological replicates. Error bars represent standard deviation. (D and E) Results of β-galactosidase assays for the pulcherrimin reporter P_{yvmC}-lacZ under shaking (24 h, D) and pellicle (48 h, E) conditions in LBGM broth without or with FeCl₃ supplementation at 0.05 or 0.1 mM. Averages are representative of three biological replicates. Error bars represent standard deviation. ns: statistically not significant. M.U., Miller units.

FIGS. 3A-D. Global transcriptome analyses reveal differentially expressed genes in the pulcherrimin mutant relative to wild type. (A) Volcano plot depicting differentially expressed genes (blue and red dots) based on RNA-Seq and global transcriptome analyses. Downregulated genes with a log 2FC of −1 or lower are depicted in red, and upregulated genes with a log 2FC of 1 or higher are depicted in blue. Black dots represent genes not differentially expressed. (B) Significantly upregulated and downregulated genes were categorized according to known or predicted function. The graph shows the number of genes that fall into each category. Blue bars correspond to upregulated genes and red bars, downregulated genes. (C-D) Heatmaps displaying significantly downregulated genes for iron acquisition/homeostasis (C) and upregulated genes related to the DNA damage response (D) in the pulcherrimin mutant relative to wild type. RNA-seq transcriptome analysis was performed in samples from pellicle biofilms (collected at 72 h). Values are the average of three biological replicates per strain.

FIGS. 4A-D. The DNA damage response is elevated in the pulcherrimin mutant. (A-B) Phase contrast and fluorescence microscopy images of the wild type (LA174, top panels, A) and pulcherrimin mutant (LA224, bottom panels, B) bearing the P_{recA}-gfp reporter for DNA damage response were taken every 24 h during pellicle biofilm development. Scale bar, 10 μm. (C) Violin plots portraying the distribution of wild-type and pulcherrimin-mutant cells according to their quantified fluorescence collected at 48 h of pellicle biofilm development in (A and B). Each dot represents a single cell. Fluorescence pixel quantification of cells was carried out by using the MicrobeJ plugin of Image J. Median values are represented by dashed horizontal lines. Solid lines represent standard deviation. There is a significant difference in the fluorescence levels between the wild type and the pulcherrimin mutant (p<0.0001, ****). (D) The pulcherrimin mutant generates more spontaneous rifampicin mutants compared with the wild type (p=0.02, *). 24 h cultures in LBGM supplemented with 0.2 mM FeCl₃ of the wild-type strain and the pulcherrimin mutant were plated separately onto rifampicin agar plates (5 μg/mL) and incubated overnight. Experiments were performed in biological triplicate and error bars represent standard deviation.

FIGS. 5A-E. The pulcherrimin mutant is more sensitive to DNA-damaging agents compared with the wild type. (A-B) Phase contrast and fluorescence microscopy images obtained from cultures of the wild type (top panels, A) and the pulcherrimin mutant (bottom panels, B) bearing the P_{recA}-gfp reporter before and after challenge with the DNA-damaging agents mitomycin C (0.25 μg/mL) or H₂O₂ (100 μM) for one hour. Scale bar, 10 μm. (C-D) Single cell fluorescence was quantified for images in A (treated with mitomycin C, panel C) and in B (treated with H₂O₂, panel D) and a violin plot was generated. Each dot in the graph represents a single cell. Median values are represented by dashed horizontal lines. Solid lines represent standard deviation. The pulcherrimin mutant (red) displayed significantly higher fluorescence than the wild type (blue) in both treatments (p<0.0001, ****). (E) Percent survival of wild-type cells after treatment with H₂O₂ (final concentration at 10 mM) for 10 minutes under shaking conditions is significantly higher when compared with the pulcherrimin mutant under the same treatment (p=0.0039, **). Average is representative of three independent experiments with a total of 8 biological replicates. Each dot represents an individual value for each replicate. Error bars represent standard deviation.

FIGS. 6A-B. Strong pulcherrimin producers and cells with significantly elevated DNA damage response in the biofilm appear to anticorrelate. (A) Wild-type B. subtilis carrying the dual reporter P_{recA}-gfp and P_{yvmC}-mkate2 (LA233) was cultured in LBGM for pellicle biofilm development and fluorescence imaging was performed on samples collected after 48 h of development. A representative image for each fluorescence channel is displayed, as well as an overlay image of both GFP and mKate2. Arrows in cyan represent cells with high GFP and low mKate2 expression, while arrows in orange represent those with high mKate2 and low GFP expression. Scale bar, 10 μm. (B) Dot plot representing the distribution of cells according to their fluorescence levels for both reporters. Each dot represents a single cell. The green circle highlights the subpopulation of cells that display high fluorescence for the P_{recA}-gfp reporter and low fluorescence for the P_{yvmC}-mkate2 reporter. The magenta circle highlights the subpopulation with high P_{yvmC}-mkate2 and low P_{recA}-gfp activities.

FIGS. 7A-B: Pulcherrimin production reduces ROS accumulation. (A) Accumulation of different ROS species (H₂O₂, peroxynitrite, and/or hydroxyl radicals) in the pulcherrimin mutant and the wild type was visualized by using a ROS detection kit. Overnight cultures of the pulcherrimin mutant and the wild type growing in LBGM supplemented with 0.2 mM FeCl₃ were harvested, cells were stained with the ROS dye, and after incubation, images were taken under a fluorescence microscope. Scale bar, 10 μm. (B) Fluorescence quantification of the cells in the images (in A) was performed using the MicrobeJ plugin of Image J. Each dot in the violin plot corresponds to a single cell. The median values are represented by dashed horizontal lines. A statistically significant difference was observed between the wild type and the pulcherrimin mutant (p<0.0001, ****).

FIGS. 8A-C: Pulcherrimin production could reduce iron toxicity by lowering iron levels. (A) The promoter activity for the bacillibactin biosynthetic operon was compared between the pulcherrimin mutant (LA148) and the wild type (YQ141) by using the transcriptional reporter P_{dhbA}-lacZ. β-galactosidase assays were performed using pellicle biofilms of the two reporter strains collected every 24 hours over the course of 4 days. (B) A mutant for the pulcherrimin transporter (ΔyvmA) was also tested for the activation of the P_{dhbA}-lacZ reporter (LA262). Pellicle biofilms of the three reporter strains were harvested after 72 h, and β-galactosidase assays were performed accordingly. Results show a significant decrease in promoter activation in ΔyvmA compared with the wild type (p=0.0417, *), but higher activation compared with the pulcherrimin mutant (p=0.0097, **). (C) A working model for the function of pulcherrimin during B. subtilis biofilm development as an iron-buffering molecule. This model shows that pulcherriminic acid molecules are produced intracellularly via two enzymatic reactions carried out by YvmC and CypX, respectively, using tRNA-charged leucine as the substrate. Pulcherriminic acid molecules are exported, via a dedicated transporter YvmA, to the extracellular environment where it binds to free ferric iron ions and turns into the reddish pigment pulcherrimin. Because iron-bound pulcherrimin is insoluble, iron depletion occurs extracellularly upon large quantities of secreted pulcherriminic acid molecules competitively chelating iron. This depletion likely results in lowered intracellular iron levels, which then leads to reduced Fenton reactions and less accumulation of DNA-damaging hydroxyl radicals. Thus, pulcherrimin production acts as an extracellular iron-buffering mechanism to reduce ROS production, consequently lowering oxidative stress and preventing DNA damage in B. subtilis.

FIGS. 9A-D. Spatiotemporal antagonistic interactions mediated through pulcherriminic acid (PA) production by biofilm-forming Bacillus subtilis against Candida albicans. A. Colony-type biofilms of C. albicans or B. subtilis WT and mutant strains were generated onto potato dextrose agar (PDA) surface, following incubation at 37° C. for 72 h. Scale bar: 0.5 cm. B. Branching of B. subtilis WT colony (black arrow) facilitates PA migration and pulcherrimin build-up (blue arrow) onto C. albicans colony. The biofilm colonies were generated by spotting microbial suspensions (2 cm apart) onto 1.5% potato dextrose agar and incubating at 37° C. for either 96 or 192 h. C. The interspecies coculture assessment showing bidirectional branching of B. subtilis biofilm enabling the pulcherrimin migration towards C. albicans colony. The biofilms were generated by spotting microbial suspensions on 1.5% potato dextrose agar and incubated at 37° C. for 72 h. The black arrowhead shows traces of pulcherrimin deposited onto C. albicans colony. D. The intra-species coculture assessment of B. subtilis WT and pulcherrimin biosynthesis mutant as a control for visualizing the specificity in branching and pigment migration. The biofilms were generated by spotting microbial suspensions onto 1.5% potato dextrose agar and incubated at 37° C. for 72 h.

FIGS. 10A-C. The pulcherrimin build-up results in compromised survivability of C. albicans. A. Coculture growth of B. subtilis WT and pulcherrimin deficient mutants with C. albicans. The biofilms were generated by spotting microbial suspensions (1.5 cm apart) on 1.5% potato dextrose agar and incubated at 37° C. for 72 h. Scale bar: 0.5 cm. Black arrow indicates pulcherrimin buildup on C. albicans. B. Assessment of cellular survival in C. albicans macrocolony using propidium iodide staining following spatiotemporal co-culture interaction. Scale bar: 100 μm. C. Viability qPCR assessment of C. albicans in co-culture with B. subtilis WT or ΔymvC ΔcypX mutant in comparison with single colony after staining with PMAxx or EMA intercalating dyes. Data is presented as dCt calculated by subtracting Ct (sample, PMAxx/EMA-treated)-Ct (sample, untreated). P>0.05 vs. the non-treated controls.

FIGS. 11A-D. The hyphal surface colonization and biofilm formation by B. subtilis induces C. albicans eradication. A. Hyphal colonization by B. subtilis cells that constitutively express green fluorescent protein (YC161) in a co-culture setup in RPMI medium. Yeast filaments are stained with calcofluor white stain. Scale bar: 20 μm. B. CFU quantitation of B. subtilis WT or sinI mutant and C. albicans cells grown in a co-culture setup. The cells were prior grown in RPMI liquid media at for 24 h and plated on LB agar for colony enumeration. The graph shows the means±SEMs of three measurements. **P<0.01 vs. the non-treated controls. C. Dissecting microscope images of B. subtilis WT or biofilm pathway and matrix mutants grown with or without C. albicans in RPMI liquid media at for 24 h. Scale bar: 20 μm. D. Microscopy images of hyphal protrusion from C. albicans macrocolony edges spotted with B. subtilis. Scale bar: 20 μm. B. subtilis was spotted to the left, while C. albicans was spotted to the right within 1.5 cm apart.

FIGS. 12A-E. Surfactin production by B. subtilis is instrumental in antagonizing C. albicans pathogenicity. A. Spatiotemporal coculture assessment of interactions between B. subtilis ΔsrfAA mutant and C. albicans. The biofilms were spotted on potato dextrose agar and at 37° C. for 72 h. Scale bar: 0.5 cm. B. Microscopic images of B. subtilis ΔsrfAA mutantgrown with or without C. albicans in RPMI liquid media at for 24 h. Scale bar: 20 μm. C. CFU quantitation of B. subtilis WT or ΔsrfAA mutant and C. albicans cells grown in a co-culture setup. The cells were prior grown in potato dextrose broth at for 24 h and plated on LB agar for colony enumeration. The graph shows the means±SEMs of three measurements. D. CFU quantitation of B. subtilis WT or ΔsrfAA mutant and C. albicans cells grown in a co-culture setup. The cells were prior grown in RPMI medium at for 24 h and plated on LB agar for colony enumeration. The graph shows the means±SEMs of three measurements. E. Survival rates of nematodes (that were prior fed with B. subtilis WT, ΔsrfAA mutant and E. coli OP50) infected with C. albicans. **P<0.01 vs. the non-treated controls.

FIGS. 13A-D. Spatiotemporal coculture assessment of interactions between B. subtilis WT and bacterial pathogens. A. Monocultures of bacterial pathogens on PDA. The biofilms were spotted on 1.5% potato dextrose agar and incubated at 37° C. for 72 h. Scale bar: 0.5 cm. B. Co-culture of B. subtilis WT and with E. coli. The biofilms were spotted on 1.5% potato dextrose agar and incubated at 37° C. for 72 h. Scale bar: 0.5 cm. Arrow indicates pulcherrimin build-up. C. Co-culture of B. subtilis WT with S. aureus. The biofilms were spotted on 1.5% potato dextrose agar and incubated at 37° C. for 72 h. Scale bar: 0.5 cm. Arrow indicates pulcherrimin build-up. D. Co-culture of B. subtilis WT with PA14. The biofilms were spotted on 1.5% potato dextrose agar and incubated at 37° C. for 72 h. Scale bar: 0.5 cm.

FIGS. 14A-D. Inhibitory effect of Bacillary pulcherrimin on biofilm formation by C. albicans. A. Biofilm formation of C. albicans in the presence or absence of purified pulcherrimin extract from WT cells (PE) as quantified by crystal violet staining. The graph shows the means±SEMs of three measurements. ***P<0.001 vs. the non-treated controls. B. Biofilms of C. albicans in the presence and absence of purified pulcherrimin from yvmC-cypX::tet srfAA::mls amyE::Pspank-yvmC-cypX, spec mutant (PE1) quantified by crystal violet staining. The graph shows the means±SEMs of three measurements. ***P<0.001 vs. the non-treated controls. C. Compound fluorescence microscopy images of C. albicans biofilms treated with purified pulcherrimin. The microtiter plates containing 24 h C. albicans biofilms were stained with SYTO™ 9. Scale bar: 200 μm. D. Differential interference contrast images of C. albicans hyphal filaments in RPMI treated with purified PE1 and PE2. Scale bar: 20 μm. Inset shows the filaments and the yeast cells in respective panels.

FIG. 15A. Effect of purified pulcherrimin (PE1) on survival of worms. P<0.05 vs. the non-treated controls.

FIG. 15B. Survival rates of nematodes (that were prior fed with B. subtilis WT, ΔsrfAA mutant and E. coli OP50) infected with C. albicans. ***P<0.001 and **P<0.01 vs. the nontreated controls.

FIG. 15C. Survival rates of nematodes (that were prior fed with B. subtilis WT, ΔcypX ΔYmvC double mutant and E. coli OP50) infected with C. albicans. ***P<0.001 and **P<0.01 vs. the non-treated controls.

FIGS. 16A-B. Graphical illustration of modes of antagonistic process for mitigating C. albicans virulence. A. The scheme depicts the production of pulcherrimin acid (PA) by B. subtilis in an iron rich environment and its subsequent migration to C. albicans. Following the PA binding of ferric iron to form red-coloured pigment pulcherrimin, it is deposited onto yeast population and compromises the pathogenicity of C. albicans. B. Schematic representation of C. albicans virulence in epithelial cells (i), which could be antagonized by probiotic bacteria through mitigating C. albicans infection (ii).

FIG. 17. Enhancing pigment production by the engineered B. subtilis strain during growth in chickpea milk (CPM). The pigment biosynthesis was induced using 1 mM IPTG. Strain 1: LA 33, with the Phyperspank-YvmC-CypX; Strain 2: LA 43, with the yvmC-cypX::tet srfAA::mls.

### DESCRIPTION OF SPECIFIC EMBODIMENTS OF THE INVENTION

The present invention, in some embodiments thereof, relates to methods of generating pulcherrimin and pulcherriminic acid and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Pulcherrimin is an iron-binding reddish pigment produced by various bacterial and yeast species. In the soil bacterium Bacillus subtilis, this pigment is synthesized intracellularly as the colorless pulcherriminic acid during biofilm formation. The biosynthesis pathway of pulcherrimin involves the activity of two key enzymes; a cyclodipeptide synthase YvmC, which catalyzes the formation of cyclic-di-leucine from two leucine-charged tRNA molecules, and a cytochrome P450 oxidase CypX, which oxidizes the cyclic dipeptide, yielding pulcherriminic acid. Once this molecule is secreted to the extracellular environment, it chelates ferric iron (Fe⁺³), turning itself into the reddish pigment pulcherrimin.

The present inventors have now shown that it is possible to genetically engineer bacteria which, in their native state, endogenously secrete pulcherriminic acid, to over-express this molecule without the need for generating biofilm. Specifically, Bacillus subtilis genetically modified to express both YvmC and CypX were shown to produce pulcherriminic acid in very large quantities in the absence of biofilm and in the absence of high concentrations of iron.

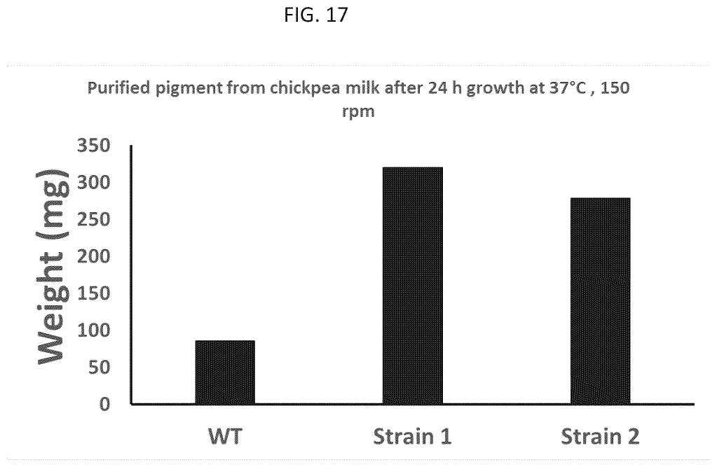

The present inventors generated a strain of Bacillus subtilis that lacked the ability to produce surfactin, another peptide-based secondary metabolite that is abundantly produced by B. subtilis, but yet was still able to produce large quantities of pulcherriminic acid upon induction, paving the way for the production of a more pure extract of pulcherriminic acid (FIG. 17).

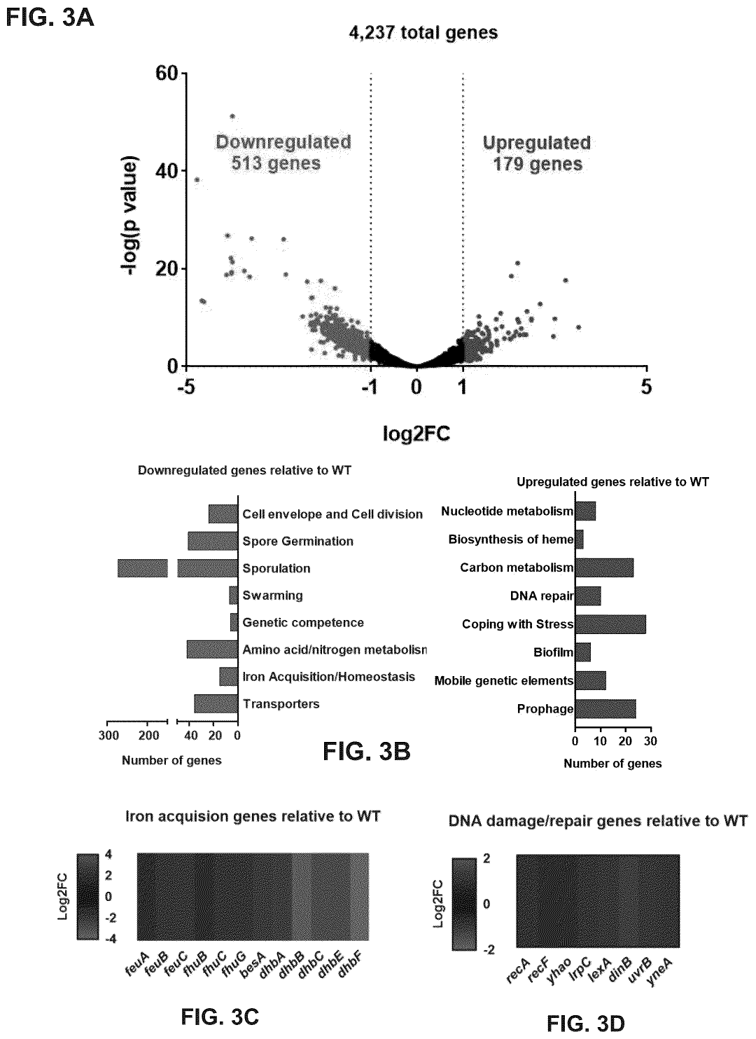

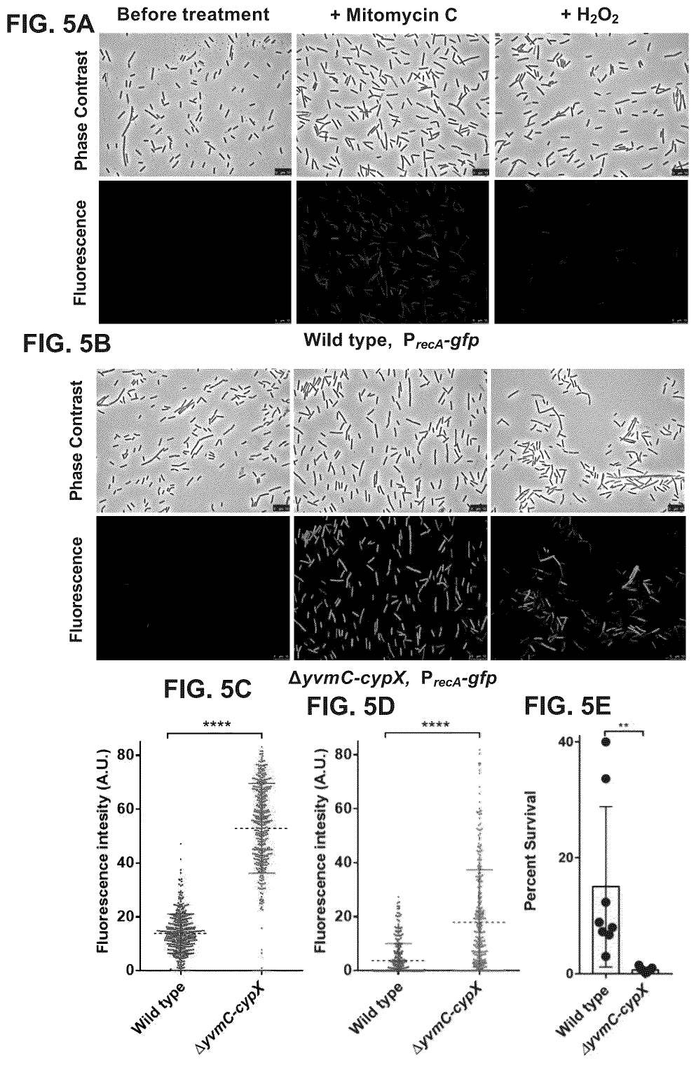

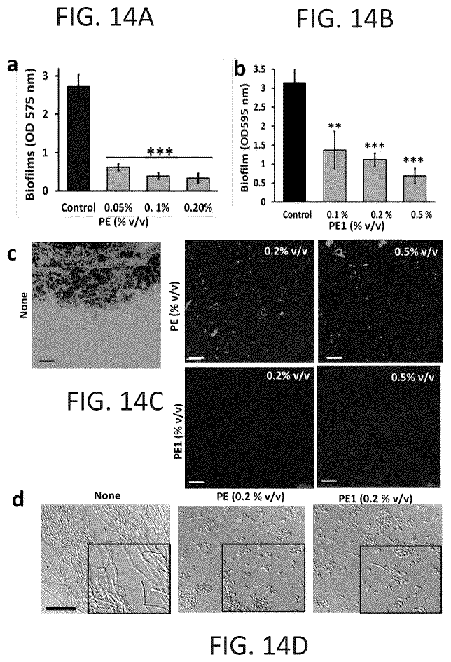

Whilst reducing the present invention to practice, the present inventors have shown that purified pulcherrimin from this strain was capable of suppressing the biofilms and hyphae of C. albicans (FIGS. 14B-D). Thus, the present inventors contemplate that purified preparations of pulcherriminic acid and/or pulcherrimin can be used as anti-fungal agents. Furthermore, the present inventors showed that pulcherrimin effects sporulation of bacteria (FIGS. 3A-B) and plays an anti-oxidative role protecting bacteria from oxidative stress (see for example FIGS. 3A-D and 4A-D and FIG. 5E). The present inventors propose that purified preparations of pulcherriminic acid and/or pulcherrimin can use used to protect probiotic bacteria and can enhance beneficial effects thereof.

Thus, according to a first aspect of the present invention, there is provided a composition of matter comprising genetically modified bacteria of the Bacillus genus which over-express cyclodipeptide synthase (YvmC) and/or cytochrome P450 oxidase (CypX), wherein the bacteria synthesize at least twice the amount of pulcherriminic acid as compared to the amount synthesized in a non-genetically modified bacteria of a corresponding strain.

In one embodiment, the bacteria that are comprised in the composition are capable of endogenously synthesizing pulcherriminic acid (i.e. in their wild-type, native form) and comprise the necessary cell machinery for its synthesis.

Bacteria belonging to the class Bacilli, includes the orders Bacillales and Lactobacillus.

In one embodiment, the bacteria are of the genus Bacillus, e.g. of the species Bacillus subtilis, Bacillus sonorensis, Bacillus licheniformis, Bacillus firmus, Bacillus megaterium, B. endophyticus, Bacillus endophyticus and Bacillus amyloliquefaciens.

According to a particular embodiment, the species is Bacillus subtilis or Bacillus licheniformis.

Exemplary strains of Bacillus species contemplated by the present invention include, but are not limited to B. paralicheniformis MS303, B. licheniformis MS310, B. paralicheniformis S127, B. subtilis MS1577, NCIB3610, Bacillus isolate BB124; B. subtilis natto, B. subtilis 168 and B. subtilis PY79.

According to a particular embodiment, the bacteria is not a strain of B. velezensis.

The genetically modified bacteria disclosed herein synthesize at least 1.5 times, at least twice, at least three times, at least four times, at least five times the amount of pulcherriminic acid as compared to the amount synthesized in a non-genetically modified bacteria of a corresponding strain (i.e. prior to genetic modification) when cultured under the same culturing conditions (e.g. in a medium comprising an identical concentration of iron).

The genetically modified bacteria of this aspect of the invention are recombinant bacteria which are genetically modified to over-express cyclodipeptide synthase (YvmC) and/or cytochrome P450 oxidase (CypX).

In one embodiment, the enzyme which is expressed in the bacteria of the present invention is a homolog and/or comprises modifications including additions or deletions of specific amino acids to the sequence (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to the native amino acid sequence of the enzyme, as determined using BlastP software of the National Center of Biotechnology Information (NCBI) using default parameters). The homolog may also refer to an ortholog, a deletion, insertion, or substitution variant, including an amino acid substitution, thereof and biologically active polypeptide fragments thereof. The homolog typically retains the enzymatic activity of the native enzyme.

Thus, YvmC may for example be a B. subtilis YvmC being encoded by a sequence as set forth in SEQ ID NO: 1 or SEQ ID NO: 2. The amino acid sequence may be at least 90% identical, at least 91% identical, at least 92% identical, at least 93% identical, at least 94% identical, at least 95% identical, at least 96% identical, at least 97% identical, at least 98% identical, at least 99% identical to SEQ ID NO: 1 or 2.
SEQ ID NO: 1 (YvmC in Bacillus subtilis NCIB3610)
MTGMVTERRS VHFIAEALTE NCREIFERRR HVLVGISPEN SRESEDYIYR LIGWAKAOFK SVSVLLAGHE AANLLEALGT PRGKAERKVR KEVSRNRRFA ERALVAHGGD PKAIHTESDF IDNKAYOLLR QEVEHAFFEQ PHERHACLDM SREAIIGRAR GVSLMMEEVS EDMLNLAVEY VIAELPFFIG APDILEVEET LLAYHRPWKL GEKISNHEFS ICMRPNQGYL IVQEMAQMLS EKRITSEG
SEQ ID NO: 2 (YvmC in Bacillus licheniformis ATCC14580)
MTELIMESKH QLFKTETLTQ NCNEILKRRR HVLVGISPEN SRFSEDYIHR LIAWAVREFQ SVSVLLAGKE AANLLEALGT PHGKAERKVR KEVSRNRRFA EKALEAHGGN PEDIHTESDE ANQTAYRNER MEVEAAFFDQ THERNACLEM SHAAILGRAR GTRMDVVEVS ADMLELAVEY VIAELPFFIA APDILGVEET LLAYHRPWKL GEQISRNEFA VKMRPNQGYL MVSEADERVE SKSMQEERV

Thus, CypX may for example be a B. subtilis CypX being encoded by a sequence as set forth in SEQ ID NO: 3 or SEQ ID NO: 4. The amino acid sequence may be at least 90% identical, at least 91% identical, at least 92% identical, at least 93% identical, at least 94% identical, at least 95% identical, at least 96% identical, at least 97% identical, at least 98% identical, at least 99% identical to SEQ ID NO: 3 or 4.
SEQ ID NO: 3 (CypX in Bacillus subtilis NCIB3610)
MSQSIKLFSV LSDQFONNPY AYFSQLREED PVHYEESIDS YFISRYHDVR YILQHPDIFT TKSIVERAEP VMRGPVLAQM HGKEHSAKRR IVVRSFIGDA LDHLSPLIKO NAENLLAPYL ERGKSDLVND FGKTFAVCVT MDMLGLDKRD HEKISEWASG VADFITSISQ SPEARAHSLW CSEQLSQYLM PVIKERRVNP GSDLISILCT SEYEGMALSD KDILALIINV LLAATEPADK TLALMIYHLL NNPEQMNDVL ADRSLVPRAI AETLRYKPPV QLIPRQLSQD TVVGGMEIKK DTIVECMIGA ANRDPEAFEQ PDVENIHRED LGIKSAFSGA ARHLAFGSGI HNCVGAAFAK NEIEIVANIV LDKMRNIRLE EDFCYAESGL YTRGPVSLLV AFDGA
SEQ ID NO: 4 (CypX in Bacillus licheniformis ATCC14580)
MNOSIKTFSV LSEQYHENPY QYFSYLRESD PVHYEESLDS YFISRYQDVR RVLONQDVFT TKSLAKRAEP VMRGPVLAQM KGKEHTAKRR IVLRRFIGES LDHLTPLIKE NAORLLAPHV EKGRIDLVND EGKTFAVCVT MDILGLDKND HQTVRNWHSG VADFITSINQ APEDREHSLK CSEQLAEYIN PIIEERRKNP GHDLISILCT SEYEGVAMSD RDIRALILNI LLAATEPADK TLALMIYHLL HHPDQMNDVI EDRTLLPQAI AETLRYKPPV QLIPROLSQD AEIGGVELKE GTTVFCMIGA ANRDPEAFED PDKENIHRSD LEVKSAFSGA ARHLAFGSGV HNCVGAGFAK TEIELVANIV IDOLKNIRLE EDFIYRETGL YTRGPVSENI REDAKH

As used herein, “% identity” and “% homology” are used interchangeably and refer to the level of nucleic acid sequence identity or amino acid sequence identity between a first nucleic acid or amino acid sequence when aligned to a second nucleic acid or amino acid sequence using a sequence alignment program. When a position in the first and the second sequences is occupied by the same nucleic acid or amino acid (e.g., if a position in the first nucleic acid sequence and the second nucleic acid sequence is occupied by cytosine), then the first and the second sequences are homologous at that position. If the term “% homology” or “% identity” is used herein without an indication of whether such homology refers to nucleic acid sequence identity or amino acid sequence identity, the term shall be interpreted as referring to nucleic acid sequence identity.

In general, identity between two sequences is calculated from the number of matching or homologous positions shared by the two sequences over the total number of positions compared. In some embodiments, the first and the second sequences are aligned in a manner to maximize % homology. In some embodiments, % homology refers to the % identity over the shorter of two sequences. In some embodiments, the % homology for a nucleic acid sequence includes intronic and/or intergenic regions. Exemplary levels of % identity include, but are not limited to, 85, 86, 87, 88, 89, 90, 91, 92, 93, 94, 95, 96, 97, 98, 99% or more sequence identity between a first and a second sequence.

Exemplary sequence alignment programs that may be used to determine % homology between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

To express the enzymes of the present invention using recombinant technology, a polynucleotide encoding the enzymes is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

The polynucleotide may further comprise ribosome binding sites to differentially control the expression level of the genes.

Nucleic acid sequences encoding the enzymes of some embodiments of the invention may be optimized for expression for a particular microorganism. Examples of such sequence modifications include, but are not limited to, an altered G/C content to more closely approach that typically found in the microorganism species of interest, and the removal of codons atypically found in the microorganism species commonly referred to as codon optimization.

As mentioned hereinabove, polynucleotide sequences of the present invention are inserted into expression vectors (i.e., a nucleic acid construct) to enable expression of the recombinant polypeptide. The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Exemplary bacterial based expression systems are disclosed in Baneyx et al., Current Opinion in Biotechnology, 1999; 10, 411-421 and Macrides et al, Microbiol Rev 1996, 60:512-538, incorporated herein by reference.

Contemplated promoters for expression in bacteria include the 1-arabinose inducible araBAD promoter (PBAD), the lac promoter, the 1-rhamnose inducible rhaP BAD promoter, the T7 RNA polymerase promoter, the trc and tac promoter, the lambda phage promoter pL, and the anhydrotetracycline-inducible tetA promoter/operator.

Approaches for controlling the abundance of the above mentioned proteins include altering the promoter [K. Hammer, I. Mijakovic, P. R. Jensen, Synthetic promoter libraries-tuning of gene expression, Trends in Biotechnology 24, 53-55 (2006)] or the ribosome binding site (RBS) [H. M. Salis, E. A. Mirsky, C. A. Voigt, Automated design of synthetic ribosome binding sites to control protein expression, Nat Biotechnol 27, 946-950 (2009); H. H. Wang et al., Programming cells by multiplex genome engineering and accelerated evolution, Nature 460, 894-898 (2009)] sequences, modulating the stability of transcripts and varying the degradation rate of the mature protein.

The bacteria may be transformed stably or transiently with the nucleic acid constructs of the present invention. In stable transformation, the nucleic acid molecule of the present invention is integrated into the bacteria genome and as such it represents a stable and inherited trait. In transient transformation, the nucleic acid molecule is expressed by the cell transformed but it is not integrated into the genome and as such it represents a transient trait.

Knock-in methods for expressing a gene in a bacteria are also contemplated.

In order to improve purity of the pulcherriminic acid preparation, down-regulation of the amount or activity of an enzyme involved in surfactin production—(e.g. srfAA) is contemplated.

In one embodiment, the gene srfAA is knocked-out.

According to a specific embodiment, the bacteria comprise at least one mutation in a gene encoding an enzyme involved in surfactin production. Preferably, the mutation brings about a down-regulation of the amount and/or activity of the product of the gene (e.g. srfAA).

Down-regulation of the amount (i.e. expression) or activity of genes in the bacteria may be effected using any method known in the art.

Methods of deleting or downregulating genes from the chromosome of bacteria are known to those of skill in the art and include homologous recombination, knock out techniques, RNAi etc.

For bacteria, methods such as P1 transduction from already existing knockout strains (KEIO collection) or via lambda-phage assisted recombination (Pkd46 system) may be used to knock-out specific genes.

According to a particular embodiment, the composition of matter is a bacterial culture which comprises a growth medium.

Examples of growth media that can be used to culture Bacillus bacteria include but are not limited to MRS medium, LB medium, LBGS medium, TBS medium, yeast extract, soy peptone, casein peptone, milk and meat peptone.

According to a particular embodiment, the growth medium comprises starch fibers of a legume of a leguminous plant

The term “legume” refers to the seeds or fruit of a leguminous plant.

Examples of leguminous plants which comprise starch fibers on which the bacteria may be cultured include plants of the genus Glycine, plants of the genus Phaseolus, plants of the genus Cicer, plants of the genus Pisum, plants of the genus Lens, plants of the genus Cajanus, plants of the genus Vicia, plants of the genus Arachis, plants of the genus Medicago, plants of the genus Neptunia, plants of the genus Trigonella, and plants of the genus Psophocarpus. Preferred examples thereof include plants of the genus Glycine, plants of the genus Phaseolus, plants of the genus Cicer, plants of the genus Pisum, plants of the genus Lens, plants of the genus Cajanus, plants of the genus Vicia, and plants of the genus Arachis. More preferred examples thereof include plants of the genus Glycine, plants of the genus Phaseolus, plants of the genus Cicer, and plants of the genus Pisum. Further preferred examples thereof include plants of the genus Glycine.

Examples of the plant of the genus Glycine include soybean (Glycine max). Examples of the plant of the genus Phaseolus include common bean (Phaseolus vulgaris). Examples of the plant of the genus Cicer include chickpea (Cicer arietinum). Examples of the plant of the genus Pisum include pea (pea sprout) (Pisum sativum). Examples of the plant of the genus Lens include lentil (Lens culinaris). Examples of the plant of the genus Cajanus include pigeon pea (Cajanus cajan). Examples of the plant of the genus Vicia include broad bean (Vicia faba). Examples of the plant of the genus Arachis include peanut (Arachis hypogaea). Examples of the plant of the genus Medicago include alfalfa (Medicago sativa). Examples of the plant of the genus Neptunia include water mimosa (Neptunia oleracea). Examples of the plant of the genus Trigonella include fenugreek (Trigonella foenum-graecum). Examples of the plant of the genus Psophocarpus include Goa bean (Psophocarpus tetragonolobus).

In a particular embodiment, the legume is chickpea.

As used herein, the phrase “starch fiber” refers to a polysaccharide comprising at least 3 sugar monomers. The size of fibers may vary between 10-1000 μm.

Starch fibers typically auto-fluoresce when visualized under a confocal laser scanning microscope. Propidium iodide (PI) staining may be used to confirm the presence of starch fibers since it selectively stains the auto-fluorescent starch particles and does not penetrate the membranes of starch granules.

Methods of releasing (or enhancing the amount of) starch fibers from legumes include heating (e.g. cooking) for an amount of time such that autofluorescence may be observed under a fluorescent microscope. Thus, for example chickpeas may be cooked for about 20 minutes to about 60 minutes.

The starch fibers may be retrieved from fresh legumes, frozen legumes, dried legumes or canned legumes.

The legumes may be treated prior to heating to enhance the process of starch fiber release. Thus, the legume may be soaked, crushed, milled and/or homogenized prior to heating.

Optionally, the heated legumes may be further treated prior to use. Exemplary treatment methods include filtration, homogenization and extraction.

Once the starch fibers are released from the legume, they may be used as part of a culture medium. The culture medium is pasteurized or sterilized.

In one embodiment, the culture medium is a chickpea milk i.e. a liquid chickpea suspension, as further described in the methods section herein below, which comprises chickpea starch fibers.

In another embodiment, the culture medium is a medium known for culturing bacillus, to which isolated (exogenous) chickpea fibers have been added. Additional methods of isolating chick pea fibers are known in the art-see for example U.S. patent application No. 20200390131, the contents of which are incorporated herein by reference.

In one embodiment, chickpea fibers are isolated using the following steps:
i. Chickpea material is subjected to an oil separation. The oil separation can be performed with a solvent such as, but not limited to, hexane, petroleum ether, or ethanol;
ii. Separation of starch and fibers is then carried out on the basis of their density.
Examples of growth media which can be used for culturing bacillus bacteria (to which the chickpea fibers may be added) include, but are not limited to LB, LBGM, milk and MRS. Additional examples of growth media are provided in US Patent Application No. 2020-0190463-A1.

Thus, the present invention contemplates growth media which are fortified with chickpea fibers (e.g. dried chickpea fibers). In one embodiment, the growth medium is chickpea milk fortified with exogenous (i.e. isolated) chickpea starch fibers.

The amount of iron in the medium may be selected according to whether preparations of pulcherrimin or pulcherriminic acid are required.

For example, in order to produce pulcherrimin, the amount of iron in the medium is contemplated to be between 0.005-0.5 mM or 0.05-0.5 mM or 0.05-0.2 mM.

In order to produce pulcherriminic acid, the amount of iron in the medium is typically below 0.05 mM or even below 0.02 mM.

In one embodiment, the amount of iron in the culture is selected such that the ratio of pulcherriminic acid:pulcherrimin is greater than 1:1, 2:1, 3:1, 4:1 or even 5:1.

In still other embodiments, the amount of iron in the culture is such that pulcherrimin is synthesized. Following removal of the medium from the culture, the iron may be removed from the pigment so as to produce pulcherriminic acid—see for example Uffen and Canale Parola., Zeitschrift fur Allg. Mikrobiologie, 9, 3, 1969, pages 231-233, incorporated herein by reference.

As mentioned, in order to generate the cell culture of this aspect of the present invention, the genetically modified bacteria are cultured in a medium under conditions effective to allow secretion of pulcerrimin/pulcherriminic acid into the medium.

In one embodiment, the medium is a liquid medium (complex or minimal).

In another embodiment, the medium is a solid medium.

In one embodiment, the conditions are such that prevent or substantially reduce formation of a biofilm (e.g. growing bacterial cells in non-static conditions such as shaking culture (e.g. 100-300 rpm) or with shear force, (e.g. at 37° C. in LB (or other non-biofilm promoting media).

The extract may be added to additional growth media in sufficient amounts such that the secretion of pulcherrimin/pulcherriminic acid is enhanced.

Other conditions of the culture that may be altered to enhance generation of pulcherrimin/pulcherriminic include, but are not limited to environmental parameters such as pH, nutrient concentration, co-culture of additional bacteria and temperature.

In one embodiment, the culturing is carried out in a bioreactor.

As used herein, the term “bioreactor” refers to an apparatus adapted to support the culture of the genetically modified bacteria.

The bioreactor may be adapted to reduce significant surface area so as to limit the formation of biofilm. The bioreactors of the invention may be adapted for continuous throughput.

The culturing of this aspect of the present invention may be carried out in the presence of additional agents that serve to increase propagation of the genetically modified bacteria. Such agents include for example acetoin.

The amount of acetoin and the timing of addition may be altered so as to promote optimal biofilm production. In one embodiment, about 0.01-5% acetoin is used. In another embodiment, about 0.01-4% acetoin is used. In another embodiment, about 0.01-3% acetoin is used. In another embodiment, about 0.01-2% acetoin is used. In another embodiment, about 0.01-1% acetoin is used. In another embodiment, about 0.01-0.5% acetoin is used.

In one embodiment, about 0.05-5% acetoin is used. In another embodiment, about 0.05-4% acetoin is used. In another embodiment, about 0.05-3% acetoin is used. In another embodiment, about 0.05-2% acetoin is used. In another embodiment, about 0.05-1% acetoin is used. In another embodiment, about 0.05-0.5% acetoin is used.

In one embodiment, about 0.1-5% acetoin is used. In another embodiment, about 0.1-4% acetoin is used. In another embodiment, about 0.1-3% acetoin is used. In another embodiment, about 0.1-2% acetoin is used. In another embodiment, about 0.1-1% acetoin is used. In another embodiment, about 0.1-0.5% acetoin is used.

The culture may comprise additional probiotic bacteria which may be co-cultured with the genetically modified bacteria. In one embodiment, the additional probiotic bacteria are capable of endogenously synthesizing pulcherrimin/pulcherriminic acid (without the need for genetic modification). In another embodiment, the additional probiotic bacteria are not capable of endogenously synthesizing pulcherrimin/pulcherriminic acid.

Particular examples of bacteria that can be included in the co-culture include Lactobacillus plantarum, L. casei, L. rhamnosus, and/or Pediococcus acidilaci.

The cultures of this aspect of the present invention are propagated for a length of time sufficient to collect a sufficient amount of pulcherrimin/pulcherriminic acid and or sufficient quantity of genetically modified bacteria.

In one embodiment, the genetically modified bacteria are cultured for at least 6 hours, 12 hours, 24 hours, 48 hours, 3 days, 4 days, 5 days, 6 days, 7 days, 8 days, 2 weeks, 3 weeks or longer to allow for sufficient quantities of the pulcherrimin/pulcherriminic acid to be secreted or a sufficient amount of genetically modified bacteria to be produced.

In one embodiment, the genetically modified bacteria are cultured at a temperature between 20-40° C., more preferably between 23-32° C.—for example at about 30° C.

According to a particular embodiment, the B. subtilis bacteria are cultured in LBGM at about 30° C.

Once sufficient quantities of genetically modified bacteria are produced, the bacteria itself may be isolated and stored. In one embodiment, the bacteria is dried (e.g. lyophilized). In another embodiment, the bacteria is frozen.

Additional compositions contemplated by the present inventors are those that are enriched in pulcherrimin/pulcherriminic acid synthesized by the genetically modified bacteria described herein. According to a particular embodiment, the composition is a conditioned medium.

Conditioned medium is the culture medium of a culture of the genetically modified bacteria (e.g. B. subtilis or B. licheniformis) following a certain culturing period. The conditioned medium includes the pulcherrimin/pulcherriminic acid secreted by bacteria in the culture.

The culture medium can be any medium suitable for culturing the bacterial cells, as described herein above.

Following accumulation of adequate pulcherrimin/pulcherriminic acid in the medium, the growth medium (i.e., conditioned medium) is separated from the bacterial cells and collected. It will be appreciated that the bacterial cells can be used repeatedly to condition further batches of medium over additional culture periods, provided that the cells retain their ability to condition the medium i.e. secrete pulcherrimin/pulcherriminic acid.

Preferably, the conditioned medium is sterilized (e.g., filtration using a 0.2 μM filter) prior to use. The conditioned medium of some embodiments of the invention may be applied directly as required or extracted to concentrate the pulcherrimin/pulcherriminic acid as further described herein above. For future use, conditioned medium is preferably stored frozen at 4° C., −20° C. or −80° C.

Additional steps may be taken to further purify the pulcherrimin/pulcherriminic acid from the culture.

In one embodiment, the pulcherrimin/pulcherriminic acid is fully isolated from the culture.

In other embodiments, a plurality (for example 1-20 or 1-10) different antimicrobial agents are isolated from the culture together with the pulcherrimin/pulcherriminic acid.

Preferably, the method for preparing the purified preparation which comprises the pulcherrimin/pulcherriminic acid involves a step of removing the culture medium from the genetically modified cells. This extracellular fraction of the liquid fermentation medium is also termed the supernatant and this fraction can be separated from the cellular fraction by e.g. centrifugation or filtration, or indeed by any other means available for obtaining a liquid fraction essentially without any bacterial cells present therein.

In particular embodiments of the invention, the purification comprises at least one size fractionation step. Preferably, this size fractionation step is performed on the extracellular fraction. This size fractionation step may ensure that every component of the composition has a molecular weight of at least a given value. The size fractionation step may be any size fraction known to the skilled person, for example ultracentrifugation, ultrafiltration, microfiltration or gel-filtration. Thus in a particular embodiment of the invention, the agent is purified from a liquid growth medium by a method involving one or more purification steps selected from the group consisting of ultracentrifugation, ultrafiltration, microfiltration and gel-filtration. Preferably, the purification step(s) are selected from the group consisting of ultrafiltration, microfiltration and ultracentrifugation, even more preferably from the group consisting of ultrafiltration and microfiltration.

Ultrafiltration is a membrane process where the membrane fractionates components of a liquid according to size. The membrane configuration is normally cross-flow wherein the liquid containing the relevant components are flowing across the membrane. Some of the liquid, containing components smaller than the nominal pore size of the membrane will permeate through the membrane. Molecules larger than the nominal pore size will be retained. The desired product may be in the retentate or the filtrate. If the ultrafiltration is performed in order to prepare a composition, wherein every agent within the composition has a molecular weight above a given value, the desired product is in the retentate. If a serial fractionation is made, the product may be in the retentate or filtrate.

Microfiltration is a membrane separation process similar to UF but with even larger membrane pore size allowing larger particles to pass through.

Gel filtration is a chromatographic technique in which particles are separated according to size. The filtration medium will typically be small gel beads which will take up the molecules that can pass through the bead pores. Larger molecules will pass through the column without being taken up by the beads.

Gel-filtration, ultrafiltration or microfiltration may for example be performed as described in R Hatti-Kaul and B Mattiasson (2001), Downstream Processing in Biotechnology, in Basic Biotechnology, eds C Ratledge and B Kristiansen, Cambridge University Press) pp 189.

In another embodiment the pulcherrimin/pulcherriminic acid in the medium may be isolated by precipitation, such as precipitation with alcohol, such as ethanol and/or chromatographic methods. This may for example be performed essentially as described in Uffen and Canale Parola., Zeitschrift fur Allg. Mikrobiologie, 9, 3, 1969, pages 231-233, incorporated herein by reference. It is also contemplated within the invention that the pulcherrimin/pulcherriminic acid are isolated by sequentially performing two or more of above-mentioned methods. By way of example the pulcherrimin/pulcherriminic acid may be isolated by first performing a size fractionation step followed by precipitation.

Other methods for isolating the pulcherrimin/pulcherriminic acid of this aspect of the present invention are also contemplated by the present inventors including but not limited to HPLC.

Preferably more than 10% of the all the components of the purified preparation derived from the genetically modified bacteria comprises pulcherrimin/pulcherriminic acid. Preferably, more than 20% of all the components of the purified preparation derived from the genetically modified bacteria comprises pulcherrimin/pulcherriminic acid. In one embodiment, more than 30% of all the components of the purified preparation derived from the genetically modified bacteria comprises pulcherrimin/pulcherriminic acid. In one embodiment, more than 40% of all the components of the purified preparation derived from the genetically modified bacteria comprises pulcherrimin/pulcherriminic acid. In one embodiment, more than 50% of all the components of the purified preparation derived from the genetically modified bacteria comprises pulcherrimin/pulcherriminic acid.

In one embodiment, the pulcherrimin/pulcherriminic acid is between 1-100% of the bacterial components in the composition of matter (per weight), between 5-100% of the bacterial bacterial components in the composition of matter, between 10-100% of the bacterial components in the composition of matter (per weight), between 15-100% of the bacterial components in the composition of matter (per weight), between 20-100% of the bacterial components in the composition of matter (per weight), between 25-100% of the bacterial components in the composition of matter (per weight), between 30-100% of the bacterial components in the composition of matter (per weight), between 35-100% of the bacterial components in the composition of matter (per weight), between 40-100% of the bacterial components in the composition of matter (per weight), between 45-100% of the bacterial components in the composition of matter, between 50-100% of the bacterial components in the composition of matter (per weight), between 55-100% of the bacterial components in the composition of matter (per weight), between 60-100% of the bacterial components in the composition of matter (per weight), between 65-100% of the bacterial components in the composition of matter (per weight), or even between 70-100% of the bacterial components in the composition of matter.

Following isolation of the pulcherrimin/pulcherriminic acid (and/or generation of the genetically modified bacteria), its presence and/or activity may be tested. In one embodiment, the anti-oxidant activity of pulcherrimin/pulcherriminic is tested. In another embodiment, the spore promoting activity of the pulcherrimin/pulcherriminic acid is tested. In still another embodiment, the ability to decrease fungal biofilm is tested. In vitro assays that can be used for confirming antimicrobial activity of the purified fractions include, for example, the addition of varying concentrations of the antimicrobial composition to paper disks and placing the disks on agar containing a suspension of the pathogen of interest. Following incubation, clear inhibition zones develop around the discs that contain an effective concentration of the antimicrobial polypeptide (Liu et al. (1994) Plant Biology 91:1888-1892, herein incorporated by reference). Additionally, microspectrophotometrical analysis can be used to measure the in vitro antimicrobial properties of a composition (Hu et al. (1997) Plant Mol. Biol. 34:949-959 and Cammue et al. (1992) J. Biol. Chem. 267:2228-2233, both of which are herein incorporated by reference). Assays that specifically measure antibacterial activity are also well known in the art. See, for example, Clinical and Laboratory Standards Institute, Guideline M7-A6, Methods for dilution antimicrobial susceptibility tests for bacteria that grow aerobically, herein incorporated by reference.

Methods of testing for the presence of pulcherrimin and pulcherriminic acid are known in the art. For example, Pulcherrimin may be indirectly measured by converting it to pulcherriminic acid using an alkali solution (e.g. 2 M NaOH solution). The amount of pulcherrimin may be determined spectrophotemetrically (e.g. 410 nm measurements).

According to another aspect of the invention, there is provided a composition comprising:
(i) a probiotic bacteria; and
(ii) pulcherriminic acid and/or pulcherrimin,
wherein the probiotic bacteria in the composition is incapable of endogenously (i.e. in the non-genetically modified strain) synthesizing pulcherriminic acid.

In a particular embodiment, the composition comprises:
(i) a probiotic bacteria; and
(ii) pulcherriminic acid, wherein the probiotic bacteria in the composition is incapable of endogenously synthesizing pulcherriminic acid.

Preferably, the compositions according to this aspect of the invention are essentially devoid of biofilm (e.g. less than 50% of the bacteria of the composition is a biofilm, less than 40% of the bacteria of the composition is a biofilm, less than 30% of the bacteria of the composition is a biofilm, less than 20% of the bacteria of the composition is a biofilm, less than 10% of the bacteria of the composition is a biofilm, less than 5% of the bacteria of the composition is a biofilm.

In one embodiment, the pulcherriminic acid and/or pulcherrimin is synthesized by the genetically modified bacteria described herein above and isolated therefrom. In another embodiment, the composition comprises the probiotic bacteria and the recombinant bacteria which are genetically modified to synthesize the pulcherriminic acid and/or pulcherrimin.

In one embodiment, the composition comprises at least twice, at least three times, at least four times, at least five times the amount of pulcherriminic acid than pulcherrimin.

The pulcherriminic acid and/or pulcherrimin is optionally present in the composition in an amount which increases the anti-oxidant properties of the probiotic bacteria. Optionally, the pulcherriminic acid and/or pulcherrimin is present in the composition in an amount which increases the ability of the probiotic bacteria to form spores.

The term “probiotic bacteria” refers to any bacteria that bring about a beneficial effect.

In one embodiment, the beneficial bacteria, when ingested promote the health of a human being. In another embodiment, the beneficial bacteria are used in industry to generate a product that is useful for human beings (e.g. methane, petroleum, insecticide etc.). In another embodiment, the beneficial bacteria are used in the food industry. In another embodiment, the beneficial bacteria are used in a silage inoculant. In still another embodiment, the beneficial bacteria are used in agriculture to support the growth of plants. In still another embodiment, the beneficial bacteria are used in bioremediation.

In one embodiment, the beneficial bacteria are live bacteria which when administered in adequate amounts confer a health benefit on the host (e.g. human).

In one embodiment the beneficial bacteria belong to the order Lactobacillies (commonly known as lactic acid bacteria (LAB)). These bacteria are Gram-positive, low-GC, acid-tolerant, generally nonsporulating, non-respiring, either rod- or coccus-shaped bacteria that share common metabolic and physiological characteristics. These bacteria produce lactic acid as the major metabolic end product of carbohydrate fermentation.

Preferably the beneficial bacteria of the Lactobacillies order are ones which grow (and are typically cultured) in MRS agar (MRS).

Exemplary contemplated genera of the order Lactobacillales include, but are not limited to Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella.

According to a preferred embodiment, the probiotic bacteria of this aspect of the present invention belong to the genus lactobacillus. Exemplary species of lactobacillus contemplated by the present invention include but are not limited to L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. bulgaricus, L. delbrueckii subsp. delbrueckii, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. hilgardii, L. homohiochii, L. iners, L. ingluviei, L. intestinalis, L. jensenii, L. johnsonii, L. kalixensis, L. kefiranofaciens, L. kefiri, L. kimchii, L. kitasatonis, L. kunkeei, L. leichmannii, L. lindneri, L. malefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. plantarum, L. pontis, L. protectus, L. psittaci, L. rennini L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae and L. zymae.

In one particular embodiment, the species of lactobacillus is L. plantarum.

The beneficial bacteria of this aspect of the present invention may generate a fermentation product. Examples of fermentation products include but are not limited to pre-biotics, biofuels, methanol, ethanol, propanol, butanol, alcohol fuels, proteins, recombinant proteins, vitamins, amino acids, organic acids (for e.g. lactic acid, propionic acid, acetic acid, succinic acid, malic acid, glutamic acid, aspartic acid and 3-hydroxypropionic acid), enzymes, antigens, antibiotics, organic chemicals, bioremediation treatments, preservatives and metabolites.

Thus, the beneficial bacteria may be genetically modified to express a beneficial polypeptide.

According to a particular embodiment, the beneficial polypeptide is an antibody (e.g. Humira, Remicade, Rituxan, Enbrel, Avastin, Herceptin).

Contemplated bacteria for the expression of antibodies include for example E. coli, Bacillus brevis, Bacillus subtilis and Bacillus megaterium.

Other beneficial bacteria contemplated by the present invention include those used as bacterial vaccines. Other contemplated beneficial bacteria are those that are useful in bioremediation. Such remediation includes heavy metals, chemical, radiation and hydrocarbon contamination.

Examples of bacteria that may be used for bioremediation are listed herein below:

Pseudomonas putida: Pseudomonas putida is a gram-negative soil bacterium that is involved in the bioremediation of toluene, a component of paint thinner. It is also capable of degrading naphthalene, a product of petroleum refining, in contaminated soils.

Dechloromonas aromatica: Dechloromonas aromatica is a rod-shaped bacterium which can oxidize aromatics including benzoate, chlorobenzoate, and toluene, coupling the reaction with the reduction of oxygen, chlorate, or nitrate. It is the only organism able to oxidize benzene anaerobically. Due to the high propensity of benzene contamination, especially in ground and surface water, D. aromatic is especially useful for in situ bioremediation of this substance.

Nitrifiers and Denitrifiers: Industrial bioremediation is used to clean wastewater. Most treatment systems rely on microbial activity to remove unwanted mineral nitrogen compounds (i.e. ammonia, nitrite, nitrate). The removal of nitrogen is a two stage process that involves nitrification and denitrification. During nitrification, ammonium is oxidized to nitrite by organisms like Nitrosomonas europaea. Then, nitrite is further oxidized to nitrate by microbes like Nitrobacter hamburgensis. In anaerobic conditions, nitrate produced during ammonium oxidation is used as a terminal electron acceptor by microbes like Paracoccus denitrificans. The result is N2 gas. Through this process, ammonium and nitrate, two pollutants responsible for eutrophication in natural waters, are remediated.

Deinococcus radiodurans: Deinococcus radiodurans is a radiation-resistant extremophile bacterium that is genetically engineered for the bioremediation of solvents and heavy metals. An engineered strain of Deinococcus radiodurans has been shown to degrade ionic mercury and toluene in radioactive mixed waste environments.

In anaerobic conditions, nitrate produced during ammonium oxidation is used as a terminal electron acceptor by microbes like Paracoccus denitrificans. The result is dinitrogen gas. Through this process, ammonium and nitrate, two pollutants responsible for eutrophication in natural waters, are remediated.

Methylibium petroleiphilum: Methylibium petroleiphilum (formally known as PM1 strain) is a bacterium capable of methyl tert-butyl ether (MTBE) bioremediation. PM1 degrades MTBE by using the contaminant as the sole carbon and energy source.

Alcanivorax borkumensis: Alcanivorax borkumensis is a marine rod-shaped bacterium which consumes hydrocarbons, such as the ones found in fuel, and produces carbon dioxide. It grows rapidly in environments damaged by oil, and has been used to aid in cleaning the more than 830,000 gallons of oil from the Deepwater Horizon oil spill in the Gulf of Mexico. Other contemplated bacteria that can be used to clean up oil include Colwellia and Neptuniibacter.

Since pulcherriminic acid has been shown to have anti-fungal properties towards yeast biofilm, they may be used to down-regulate the amount of fungus in a biofilm.

Thus, according to another aspect of the present invention there is provided a method of breaking down a biofilm of yeast comprising contacting the biofilm with an effective amount of a purified preparation of pulcherriminic acid or the compositions described herein, thereby breaking down the biofilm of yeast.

As used herein the term “contacting” refers to the positioning of the pulcherriminic acid or composition of the present invention such that they are in direct or indirect contact with the fungal cells of the biofilm. Thus, the present invention contemplates both applying the compositions of the present invention to a desirable surface and/or directly to the yeast cells.

Contacting surfaces with the compositions described herein can be effected using any method known in the art including spraying, spreading, wetting, immersing, dipping, painting, ultrasonic welding, welding, bonding or adhering. The agents of the present invention may be attached to a solid surface as monolayers or multiple layers.

The present invention envisages coating a wide variety of surfaces with the agents of the present invention including fabrics, fibers, foams, films, concretes, masonries, glass, metals, plastics, polymers, and like.

An exemplary solid surface that may be coated with the agents of the present invention is an intracorporal or extra-corporeal medical device or implant.

An “implant” as used herein refers to any object intended for placement in a human body that is not a living tissue. The implant may be temporary or permanent. Implants include naturally derived objects that have been processed so that their living tissues have been devitalized. As an example, bone grafts can be processed so that their living cells are removed (acellularized), but so that their shape is retained to serve as a template for ingrowth of bone from a host. As another example, naturally occurring coral can be processed to yield hydroxyapatite preparations that can be applied to the body for certain orthopedic and dental therapies. An implant can also be an article comprising artificial components.

Thus, for example, the present invention therefore envisions coating vascular stents with the compositions of the present invention. Another possible application of the compositions of the present invention is the coating of surfaces found in the medical and dental environment.

Surfaces found in medical environments include the inner and outer aspects of various instruments and devices, whether disposable or intended for repeated uses. Examples include the entire spectrum of articles adapted for medical use, including scalpels, needles, scissors and other devices used in invasive surgical, therapeutic or diagnostic procedures; blood filters, implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherally insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, aneurysm repair devices, heart valves, artificial joints, artificial larynxes, otological implants), anastomotic devices, vascular catheter ports, clamps, embolic devices, wound drain tubes, hydrocephalus shunts, pacemakers and implantable defibrillators, and the like. Other examples will be readily apparent to practitioners in these arts.

Surfaces found in the medical environment include also the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilized drugs in nebulizers and of anesthetic agents. Also included are those surfaces intended as biological barriers to infectious organisms in medical settings, such as gloves, aprons and face shields. Commonly used materials for biological barriers may be latex-based or non-latex based. Vinyl is commonly used as a material for non-latex surgical gloves. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered.

Other surfaces related to health include the inner and outer aspects of those articles involved in water purification, water storage and water delivery, and those articles involved in food processing. Thus the present invention envisions coating a solid surface of a food or beverage container to extend the shelf life of its contents.

Surfaces related to health can also include the inner and outer aspects of those household articles involved in providing for nutrition, sanitation or disease prevention. Examples can include food processing equipment for home use, materials for infant care, tampons and toilet bowls.

In addition, the agents of the present invention may have veterinary applications including disinfection of animal cages, coops or homes.

It will be appreciated that since the compositions of the present invention have antifungal activity, the present invention contemplates use thereof for treating fungal infection in a mammalian subject (e.g. humans). According to a particular embodiment, the infection is a Candida albicans infection.

According to one embodiment, the infection is an acute infection.

According to another embodiment, the infection is a chronic infection.

According to one embodiment, the agents are used to treat a topical infection (i.e. infection of the skin) and are provided in a topical formulation.

According to another embodiment, the agents are used to treat an infection inside the body. In this case, the agents may be provided ex vivo or in vivo.

Accordingly, the present invention contemplates contacting fungal cells with the isolated pulcherriminic acid or when it is part of a pharmaceutical composition.

In one embodiment, the pharmaceutical compositions of the present invention are administered to a subject in need thereof in order to prevent or treat a fungal infection.

As used herein, the term “subject in need thereof” refers to a mammal, preferably a human subject.

As used herein, the term “treating” refers to curing, reversing, attenuating, alleviating, minimizing, suppressing or halting the deleterious effects of the fungal infection.

The phrase “pharmaceutical composition”, as used herein refers to a preparation of one or more of the active ingredients described herein with other chemical components such as physiologically suitable carriers and excipients. The purpose of a pharmaceutical composition is to facilitate administration of a compound to an organism.

As used herein the term “active ingredient” refers to the agents of the present invention accountable for the intended biological effect (e.g. pulcherriminic acid).

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences”, Mack Publishing Co., Easton, PA, which is herein fully incorporated by reference and are further described herein below.

It will be appreciated that the agents of the present invention can be provided to the individual with additional active agents to achieve an improved therapeutic effect as compared to treatment with each agent by itself.

Exemplary additional agents include antibiotics (e.g. rifampicin, chloramphenicol and spectinomycin), antibacterial peptides, antivirals, antifungals etc.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

The preparation of the present invention may also be formulated as a topical compositions, such as a spray, a cream, a mouthwash, a wipe, a foam, a soap, an oil, a solution, a lotion, an ointment, a paste and a gel.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In further embodiments, the pulcherriminic acid is formulated to treat a fungal infection of a plant.

In a particular embodiment, the pulcherriminic acid is formulated with an agriculturally acceptable carrier.

As used herein the term “agriculturally acceptable carrier” refers to a material that facilitates application of the bacteria (or agent isolated therefrom) to the intended target, which may be for example a plant, a plant material, compost, earth, surroundings or equipment, or that facilitates storage, transport or handling. Carriers used in compositions for application to plants and plant material are preferably non-phytotoxic or only mildly phytotoxic. A suitable carrier may be a solid, liquid or gas depending on the desired formulation. In one embodiment the carriers include polar liquid carriers such as water, mineral oils and vegetable oils. In one embodiment the carrier enhances the stability of the active ingredient as described herein.

The carrier can include a dispersant, a surfactant, an additive, water, a thickener, an anti-caking agent, residue breakdown, a composting formulation, a granular application, diatomaceous earth, an oil, a coloring agent, a stabilizer, a preservative, a polymer, a coating, or a combination thereof. One of ordinary skill in the art can readily determine the appropriate carrier to be used taking into consideration factors such as a particular bacterial strain, plant to which the bacteria is to be applied, type of soil, climate conditions, whether the bacteria is in liquid, solid or powder form, and the like.

The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof.

The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination thereof.

The anti-caking agent can include a sodium salt such as a sodium sulfite, a sodium sulfate, a sodium salt of monomethyl naphthalene sulfonate, a sodium salt of dimethyl naphthalene sulfonate, or a combination thereof; or a calcium salt such as calcium carbonate, diatomaceous earth, or a combination thereof.

Exemplary agriculturally acceptable carriers include, but are not limited to, vermiculite, charcoal, sugar factory carbonation press mud, rice husk, carboxymethyl cellulose, peat, perlite, fine sand, calcium carbonate, flour, alum, a starch, talc, polyvinyl pyrrolidone, or a combination thereof.

Bacillus cultures which synthesize pulcherriminic acid (e.g. the genetically modified bacilli described herein) can be prepared as solid, liquid, emulsion or powdered formulations as is known in the art. The cultures of the present invention can be formulated as a seed coating formulation, a liquid formulation for application to plants or to a plant growth medium, or a solid formulation for application to plants or to a plant growth medium.

Examples of agents that promote the growth of a plant include a fertilizer, a micronutrient fertilizer material, an insecticide, a herbicide, a plant growth regulator, an acaricide, a rodenticide, a fungicide, a nutrient, a molluscicide, an algicide, a pesticide, a fungal inoculant, or a combination thereof.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

### Bacterial Strains and Media

TABLE 1
Strains, plasmids, and oligonucleotides

| | Description |
|---|---|
| Strains | |
| 3610 | an undomesticated strain of B. subtilis, capable of forming |
| | biofilms |
| DH5α | E. coli strain for molecular cloning |
| PY79 | a competent laboratory strain of B. subtilis used for genetic |
| | manipulation |
| YC800 | ΔyvmC-cypX::tet^{R} in 3610 |
| LA11 | amyE::P_{yvmC}-lacZ, chl^{R} in 3610 |
| LA20 | amyE::PyvmC-gfp, chl^{R} in 3610 |
| LA33 | ΔyvmC-cypX::tet^{R} amyE::Pₛₚₐₙₖ-yvmC-cypX, spec^{R} in 3610 |
| YQ141 | amyE::P_{dhbA}-lacZ, spec^{R} in 3610 |
| LA148 | ΔyvmC-cypX::tet^{R} amyE::P_{dhbA}-lacZ, spec^{R} in 3610 |
| LA174 | amyE::P_{recA}-gfp, spec^{R} in 3610 |
| LA224 | ΔyvmC-cypX::tet^{R} amyE::P_{recA}-gfp, spec^{R} in 3610 |
| LA233 | amyE::P_{recA}-gfp, spec^{R} lacA::P_{yvmC}-mkate2, erm^{R} in 3610 |
| LA208 | ΔyvmA::kan^{R} in 168 |
| LA247 | ΔyvmA::kan^{R} in 3610 |
| LA262 | ΔyvmA::kan^{R}. amyE::P_{dhbA}-lacZ, spec^{R} in 3610 |
| Plasmids | |
| LA2 | pYC121, amyE::P_{yvmc}-gfp, amp^{R} in DH5α |
| LA9 | pDG268, amyE::P_{yvmC}-lacZ, amp^{R} in DH5α |
| LA22 | pDR111, amyE::Pₛₚₐₙₖ-yvmC-cypX, amp^{R} in DH5α |
| LA72 | pYC121, amyE::P_{yvmC}-mkate2, amp^{R} in DH5α |
| LA97 | pDG268, amyE::P_{katE}-lacZ, amp^{R} in DH5α |
| LA163 | pYC211, amyE::P_{recA}-gfp, amp^{R} in DH5α |
| LA186 | pDR183, lacA::P_{yvmC}-mkate2, amp^{R} in DH5α |
| pDG268 | amyE integration vector with a promoter-less lacZ, spec^{R}, |
| | amp^{R} |
| pDR111 | amyE integration vector with an IPTG-inducible promoter, |
| | spec^{R}, amp^{R} |
| pDR183 | lacA integration vector, erm^{R}, amp^{R} |
| pYC121 | amyE integration vector with a promoter-less gfp, chl^{R}, amp^{R} |
| pYC211 | amyE integration vector with a promoter-less gfp, spec^{R}, |
| | amp^{R} |
| pYC251 | amyE integration vector with an IPTG-inducible promoter |
| | mkate2, chl^{R}, amp^{R.} |
| pDG1730 | amyE integration vector with amp^{r}, spec^{r}, and erm^{r} |

Table 1 lists all the strains and plasmids utilized in this Example. Bacillus subtilis NCIB 3610, B. subtilis PY79, Escherichia coli DH5α and derived strains were cultured in lysogeny broth (LB) (10 g/L tryptone, 5 g/L yeast extract, and 10 g/L NaCl) at 37° C. Colony and pellicle biofilms were grown statically at 30° C. in LBGM (LB broth supplemented with final concentrations of 1% glycerol (v/v) and 100 μM MnSO₄) broth or agar (1.5% w/v), respectively. When needed, antibiotics were applied at the following concentrations: 100 μg/ml of spectinomycin, 1 μg/ml of erythromycin, 10 μg/ml of tetracycline, 50 μg/ml of kanamycin, and 10 μg/ml of chloramphenicol for B. subtilis. All E. coli DH5α strains were grown in LB broth supplemented with 100 μg/ml of ampicillin.

### Bacterial Strain Construction

The single insertional deletion mutant yvmA::kan (BKE40190) used herein (listed in Table 1) was acquired from the Bacillus Genetic Stock Center as a B. subtilis 168 background. The mutation was introduced into NCIB3610 through transformation of genomic DNA, and transformants were selected for on LB agar with kanamycin, generating strain LA247. The strain YC800 (yvmC-cypX::tet) was built using long-flanking homology PCR. The primers yvmC-P1, yvmC-P2, cypX-P1, and cypX-P2 (Table 1) were used to perform the PCR reactions. The final purified PCR product was transformed into PY79, and transformants were selected on specific antibiotic plates. Confirmation of deletion mutation was performed through PCR of the locus and Sanger sequencing.

To construct the plasmid pYC211, the DNA fragment containing the gfp gene was cut from pYC121 and cloned into the HindIII and BamHI sites of pDG1730. The plasmids bearing gfp or lacZ reporters LA2, LA9, LA97 and LA163 (in DH5α) were designed using the following protocol. First, the promoter region of interest (or coding sequence) was PCR amplified from NCIB3610 using forward and reverse primers (listed in table 1). The PCR product was purified after confirmation of successful amplification through agarose gel electrophoresis. Purified PCR and plasmid of interest were double digested separately using the same restriction enzymes (NEB), and ligated together using T4 DNA ligase (Invitrogen). The ligation reaction was transformed into chemically competent DH5α and, after overnight incubation of plates, colonies were picked. Promoter region insertion into the plasmid was confirmed through Sanger sequencing and DH5α strain was miniprepped to obtain enough plasmid mass for transformation into PY79. After selecting positive colonies for PY79 transformation through amylase production screening on starch agar plates, the genomic DNA of PY79 was isolated and transformed into NCIB3610, generating strains LA11, LA20, and LA174. Strains LA33 and LA224 were designed by transforming genomic DNA from PY79 containing the reporter of interest into a ΔyvmC-cypX background strain. Strains LA148 and LA262 were made by transforming genomic DNA from YQ141 into ΔyvmC-cypX and ΔyvmA backgrounds, respectively, and selecting for spectinomycin resistant colonies and lack of amylase production. Lastly, the dual reporter strain LA233 was constructed using two cloning steps. First, the LA2 plasmid was digested using restriction enzymes to remove the gfp coding sequence. Next, the mkate2 gene was amplified from pYC251 using primers mkate2-F and mkate2-R, PCR purified, and cloned into the gfp-less plasmid to make LA72. LA72 was then digested using restriction enzymes and the DNA for the P_{yvmC}-mkate2 reporter sequence was gel purified and ligated into pDR183 plasmid (LA186). This plasmid was transformed into PY79 and the genomic DNA from a confirmed colony was transformed into LA174. Selection of colonies for both spectinomycin and erythromycin was performed, and the final construct generated was the dual reporter strain LA233.

Biofilm assay: Prior to colony biofilm inoculation, cells were grown to exponential phase in LB broth. 2 μL of each cell culture were spotted onto LBGM 1.5% agar (w/v) and supplemented with 1 mM Isopropyl β-D-1-thiogalactopyranoside (IPTG). For pellicle biofilms, cells were grown to exponential phase in LB broth and 3 μL of each culture was inoculated into 3 mL LBGM broth (1:1000 dilution) in a 12-well microtiter plate (Corning, NY, USA). Both colony and pellicle biofilm plates were incubated under static conditions at 30° C. Pictures of colony biofilms were taken every 24 h using a Sony NEX-7 camera.

Cell preparation and fluorescence microscopy: Pellicle biofilm cells were primarily broken down through vigorous pipetting. 3 mL of each cell suspension were transferred to 15 mL conical tubes (Fisher Scientific) and samples were mildly sonicated using a Sonifier® cell disruptor (Heat Systems-Ultrasonics, Inc.) at the 1.5 output scale for 30 seconds to separate cells from the extracellular matrix. 1 mL of cell suspension was transferred to 1.5 mL microcentrifuge tubes (Fisher Scientific), spun down at 14,000 rpm for 1 min, washed three times with phosphate-buffered saline (PBS), and resuspended in a final volume of 100 μL of PBS. Glass coverslips were previously treated with a sterile solution of 0.01% (v/v) poly-D-lysine before four microliters of each cell suspension were added onto glass microscope slides (Fisher Scientific). Images of cells were captured using a Leica DFC3000 G camera on a Leica AF6000 fluorescence microscope. Excitation and emission wavelengths used to visualize cells expressing GFP (and cells stained with the ROS dye) were 450-490 nm and 500-550 nm, respectively. The excitation and emission wavelengths used to visualize cells expressing mKate2 were 540 to 580 nm and 610 to 680 nm, respectively. By measuring wild-type cells without fluorescence, non-specific background fluorescence was defined. Cells grown under shaking conditions were prepared the same way as described above besides the sonication step. Each image obtained is representative of three biological replicates.

Microplate growth curves: Each strain was grown overnight in 3 mL of LB broth with shaking at 200 rpm at 37° C. The next day, cells were sub-cultured 1:100 in fresh LB broth at a final volume of 150 μL, and transferred into a 96-well tissue culture microplate (Corning, NY, USA). For cultures where IPTG was supplemented, the final concentration added to the medium was 1 mM. For the mitomycin C MIC test, a final concentration of 0.1, 0.25, 1, 2, 3, 4, or 5 μg/mL was added to the respective wells. For the H₂O₂ MIC test, a final concentration of 5, 10, 50, 100, 250, 500 or 1000 μM was added to the respective wells. Bulk culture growth by cell optical density (OD600 nm) was measured for 16 hours at 15-minute time point intervals in a BioTek plate reader under shaking conditions at 37° C. Averages and standard deviations were calculated, and data was plotted as OD₆₀₀ values vs time using Graphpad Prism 9. Three independent experiments were performed, and for each experiment, three biological replicates per strain were used. Statistical analysis was performed using Student's t-test on Graphpad Prism 9.

β-Galactosidase activity assays: LBGM medium was used to cultivate pellicle biofilms of each strain as described above. At each time point, biofilms were softly sonicated, and either 1 mL or 100 μL of cell suspension were harvested in 2 mL microcentrifuge tubes (Fisher Scientific). Cells were pelleted by centrifugation at 14,000 rpm for 1 minute, the supernatant discarded, and samples stored at −80° C. until use. Pellets were thawed at room temperature for 5 minutes, then resuspended in 1 mL of Z buffer (final concentrations: 200 mg/ml of lysozyme, 40 mM NaH₂PO₄, 60 mM Na₂HPO₄, 1 mM MgSO₄, 10 mM KCl, and 38 mM-mercaptoethanol). Samples were incubated at 30° C. for 15 minutes followed by addition of 200 μL of 4 mg/ml o-nitrophenyl-D-galactopyranoside (ONPG) to each tube to start the reaction. After a faint yellow color was observed in the tubes, 500 μL of 1M Na₂CO₃ solution was added to stop the reaction and the reaction time was recorded. To get rid of any cell debris, samples were spun down for 10 minutes at 5,000 rpm. The supernatants were transferred to 1 mL plastic cuvettes (Fisher Scientific) and absorbance readings at 420 and 550 nm were taken for each sample using a Bio-Rad SmartSpec 3000 spectrophotometer. The formula used to determine the β-galactosidase-specific activity was the following: [OD₄₂₀−1.75 (OD₅₅₀)/(time×volume in mL×OD600)]×1,000. Results were plotted as bar graphs and statistical significance analyses were performed using Student t-test on Graphpad Prism 9. Each experiment was performed three times, each with three biological triplicates per strain.

Fluorescence quantification of single cells: The Microbe J plugin of the ImageJ software was used to quantify the fluorescence intensity for each cell from images obtained by fluorescence microscopy. A minimum of 200 cells per strain and per treatment were measured. After quantifications, fluorescence values were plotted in a violin plot, where each dot represents a single cell, and the Student t-test was used to determine statistical significance amongst strains or treatments on Graphpad Prism 9.

Determination of mutation frequency: In order to determine the mutation frequency between the wild type and the pulcherrimin mutant, a protocol based on a previously published study 58 was utilized. Wild type and the pulcherrimin mutant were cultured separately in 250 mL flasks containing 50 mL LBGM broth supplemented with 0.2 mM FeCl₃ for strong pigment production. After 24 hours of growth, aliquots of each culture were serially diluted and plated on LB agar for CFU quantification. For mutation frequency determination, a 1.5 mL aliquot of each culture was pelleted and resuspended in 150 μL of LB broth. The entire volume was plated onto LB agar plates supplemented with rifampicin to a final concentration of 5 μg/mL. Plates were incubated at 37° C. overnight and spontaneous rifampicin mutant colonies were counted the next day. Data visualization and statistical analysis were performed using Graphpad Prism 9.

Challenging cell cultures with DNA damaging agents: Thirty microliter (μL) of overnight cultures of the wild type and pulcherrimin mutant bearing the P_{recA}-gfp reporter were subcultured in test tubes containing 3 mL of LB broth each (1:100 dilution). Tubes were placed in the shaker for 2 hours at 37° C. and 200 rpm. After that, experimental tubes were challenged with either mitomycin C or H₂O₂ at final concentrations of 0.25 μg/mL or 100 μM, respectively. Control tubes did not have either DNA-damaging molecule added. Tubes were placed back in the shaker for one more hour, then 1 mL of cells were harvested for each sample. Cells were washed with PBS three times and imaged under a fluorescence microscope. Fluorescence quantification was performed, data was plotted and statistical analysis was carried out using Student's t-test on Graphpad Prism 9. For each strain and treatment, a total of three biological replicates were tested.

Killing assay using hydrogen peroxide: Cultures of the wild type and pulcherrimin mutant were grown overnight at 37° C. and 200 rpm in 50 mL LBGM supplemented with FeCl₃ at the final concentration of 0.2 mM. On the next day, the volume of each culture was split into two new flasks. The first flask had H₂O₂ added at the final concentration of 10 mM. An equal volume of sterile water was added instead of H₂O₂ to the control flask. Flasks were placed back into the shaker for ten minutes, then 200 μL of cells were collected from each sample and serially diluted from 10-1 to 10-10 in sterile LB broth. Ten microliter (μL) of each serial dilution were plated on LB 1.5% agar, and plates were incubated overnight at 37° C. Colony forming units (CFUs) were counted the next day. Percent cell survival was calculated by dividing the number of CFUs counted for the experimental flask by the number of CFUs in the control sample, multiplied by 100. Data was plotted as bar graphs and statistical significance analyses were performed using Student's t-test on Graphpad Prism 9. Each experiment was performed three times, each in biological triplicate per strain.

Total ROS detection: Overnight cell cultures of the wild type and pulcherrimin mutant were inoculated in biological triplicate in 50 mL of LBGM supplemented with FeCl₃ at the final concentration of 0.2 mM. On the next day, 1 mL of each culture was harvested, washed with PBS three times, and incubated with the Oxidative Stress Detection Reagent from the ROS-ID® Total ROS Detection Kit (Enzo Life Sciences) according to the manufacture's protocol. Cells were imaged using fluorescence microscopy, and fluorescence quantification, data representation on a violin plot and statistical significance were performed as described above. The Oxidative Stress Detection Reagent is a cell-permeable, non-fluorescent molecule that directly reacts with a variety of reactive oxygen species, including hydrogen peroxide, peroxynitrite, and hydroxyl radicals, producing green fluorescence result indicative of ROS presence. Therefore, there is a positive correlation between the intracellular levels of ROS and the fluorescence intensity observed.

Global transcriptome profiling by RNA-Seq: Pellicle biofilms of the wild type and pulcherrimin mutant were grown in LBGM as described above, and samples were harvested after 72 hours of development. Biofilms were softly sonicated, washed with PBS three times, measured for absorbance at OD₆₀₀ nm, and a total of 10⁹ cells were pelleted through centrifugation. Cells were then incubated with RNAprotect® Bacteria Reagent (Qiagen) according to manufacturer's protocol, pelleted again, snap frozen in liquid nitrogen, and stored at −80° C. until ready to ship. Genewiz (South Plainfileld, NJ) carried out the steps of RNA extraction, rRNA depletion, RNA fragmentation, quality control, library preparation, and sequencing through Illumina HiSeq 2×150 bp. Sequence trimming and mapping were performed using Geneious Prime. The reference genome used to map the reads and to assess differential gene expression was NZ_CP020102 (Bacillus subtilis subsp. subtilis str. 168) from NCBI. The accession number for the reference sequence used for NCIB3610 plasmid pBS32 analysis was CP020103 from NCBI.

Measurements of pulcherrimin and pulcherriminic acid: Pulcherrimin was indirectly measured by converting it to pulcherriminic acid using 2M NaOH solution. Briefly, 1 mL of each overnight LBGM shaking culture (with or without supplemented FeCl₃) was spun down for one minute at 15,000 g to pellet cells and insoluble pulcherrimin. The pellet was washed twice and resuspended in 1 mL 1×PBS. Following this, 300 μL of a 2M NaOH stock solution was added and tubes were mixed by inversion ten times until all the pulcherrimin was converted from a reddish to a yellow color (pulcherriminic acid). Samples were spun down for 1 minute at 15,000×g and 1 mL of supernatant was added to a cuvette. Following this, 410 nm measurements were performed using a Bio-Rad SmartSpec™ 3000 spectrophotometer. OD600 nm measurements of each overnight culture were also obtained for normalization of readings. Cultures of the pulcherrimin mutant grown overnight in LBGM at different FeCl₃ concentrations were used as blanks for the assay. For the spent supernatant measurements of total pulcherriminic acid, each sample was first 10-fold diluted in 1×PBS, followed by 410 nm measurements. Spent supernatants of the pulcherrimin mutant with or without added FeCl₃ were used as blanks for the assay.

### Results

Biofilm Conditions Promote Strong Production of Pulcherrimin, an Iron Chelator in B. subtilis.

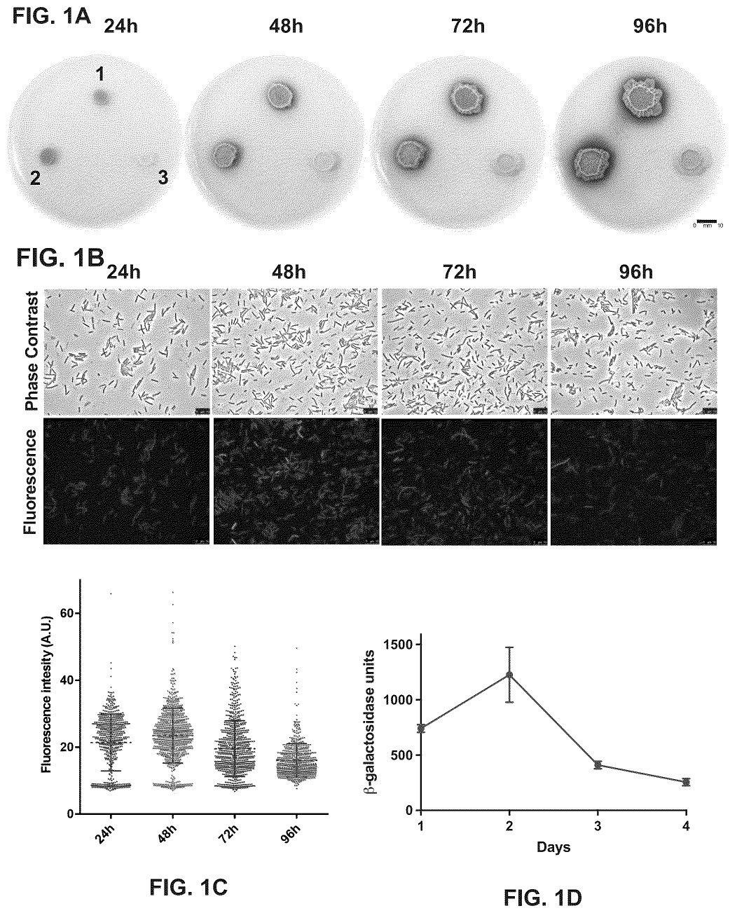

To study the red-colored, iron-binding pigment pulcherrimin, a mutant strain in B. subtilis (ΔyvmC-cypX, YC800) was constructed that abolished the production of this iron chelator, as well as a complementation strain (LA33, yvmC-cypX under the control of an IPTG-inducible promoter was integrated at the amyE locus in YC800). Cells of the pulcherrimin mutant, the wild-type strain NCIB3610 (abbreviated as wild type hereafter), and the complementation strain were spotted on the iron-supplemented biofilm-inducing media LBGM+0.2 mM FeCl₃. After incubation for 48 hours, a reddish halo surrounding the colony biofilms was clearly observed in both the wild-type and the complementation strains, but completely absent in the mutant (FIG. 1A). This suggests that the main component of the reddish pigment is pulcherrimin. Overproduction or loss of pulcherrimin did not impact growth of B. subtilis cells since no difference in growth was observed among the wild-type, the pulcherrimin-mutant, and the complementation strains.

Pulcherrimin production increased over time during biofilm development (FIG. 1A).

Global Transcription Profiling Suggests that Pulcherrimin Regulates Genes Involved in Iron Homeostasis and DNA Damage Response.

To have a better understanding of transcriptional regulation, the global transcriptome of the pulcherrimin mutant compared with the wild type under biofilm conditions was compared. Pellicle biofilms of the wild type and the mutant were grown in LBGM. Pellicles were collected after 72 hours. Global transcription profiling was performed using RNA-Seq. Principal Component Analysis (PCA) suggested that in general, the wild-type replicates clustered separately from the pulcherrimin-mutant replicates. A cut-off of log 2 fold change (log 2FC) of +/−1 for significantly up- and down-regulated genes, respectively was applied. A volcano plot was generated where a total of 4,237 genes retrieved from the RNA-Seq analysis could be observed, 513 of which significantly downregulated, and 179 upregulated in the pulcherrimin mutant (FIG. 3A). Both sets of genes were categorized according to known or predicted function, and the results are summarized in FIG. 3B. In the pulcherrimin mutant, a much higher number of upregulated genes related to general stress response (“Coping with Stress”, 28 genes) were observed, as well as DNA damage response (“DNA repair”, 10 genes). On the other hand, a significant number of genes related to iron acquisition/homeostasis (15 genes) were downregulated in the pulcherrimin mutant. More strikingly, hundreds of sporulation genes were downregulated in the pulcherrimin mutant, implying that pulcherrimin has an influence on sporulation during biofilm development in B. subtilis. Lastly, bioinformatic analysis was performed on the raw RNA-seq data using the reference sequence for the pBS32 plasmid from NCIB3610. Among the total 96 genes included in the analysis, only 4 genes were significantly downregulated and 5 upregulated.

Pulcherrimin is known to be an iron chelator. Previous studies have shown that secreted pulcherrimin caused localized iron depletion in the media. Results from the transcriptome profiling show that many iron homeostasis genes were downregulated in the pulcherrimin mutant, including the entire bacillibactin biosynthetic operon (dhbABCEF), bacillibactin transport genes (feuABC, fhuBCG) and the bacillibactin esterase gene besA (FIG. 3C). Bacillibactin is the main iron-scavenging molecule in B. subtilis, and it possesses extremely high affinity to iron ion. BesA is a bacillibactin esterase, which catalyzes the hydrolysis of iron from bacillibactin and its release intracellularly. Lastly, the operons feuABC and fhuBCG are responsible for uptake of bacillibactin bound to iron from the extracellular environment 54. A plausible explanation for the above results is that in the wild-type biofilm (day 3 pellicle), cells experience iron limitation stress due to chelation of ferric iron by pulcherrimin in the media, thus increasing the expression of those iron-scavenging pathway genes. Indeed, localized iron depletion caused by pulcherrimin production has already been reported in the previously published study. This result also supports the idea that pulcherrimin is not likely a siderophore used for iron acquisition by B. subtilis.

Among the upregulated genes in the pulcherrimin mutant, a significant number of DDR genes were observed. Many of these genes are controlled by the DDR regulator LexA (recA, yhaO, lexA, dinB, yneA and uvrB, FIG. 3D), and are part of the DDR pathway that is upregulated when cells experience DNA damage.

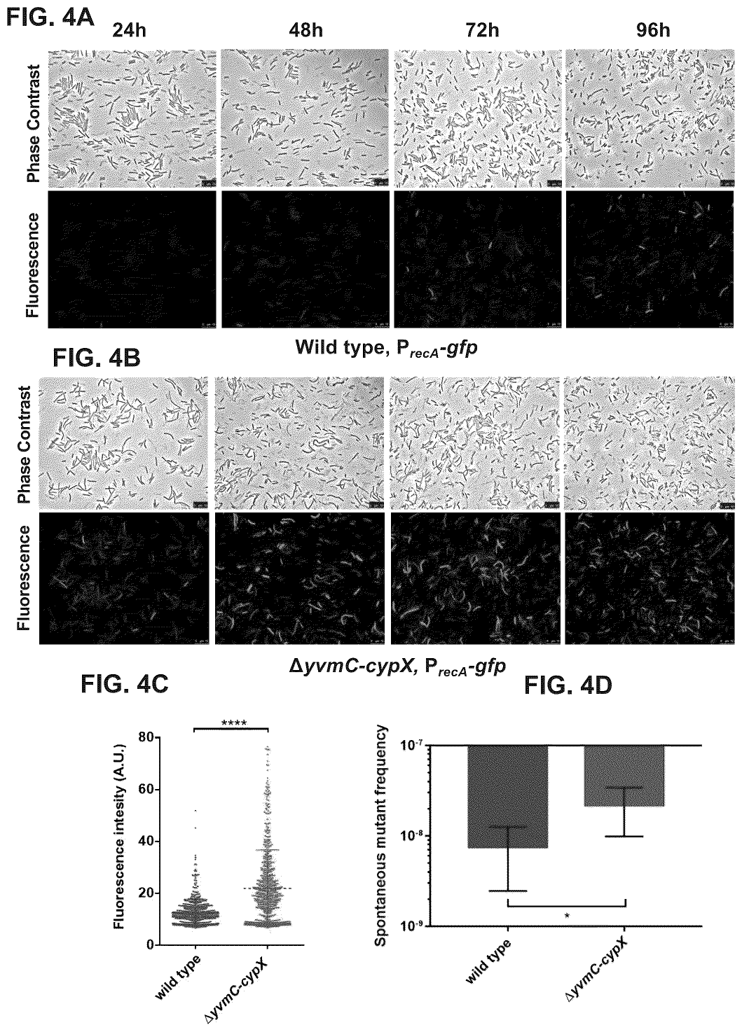

Pulcherrimin protects cells from DNA damage. The RNA-Seq results indicated that multiple DDR genes were upregulated in the pulcherrimin mutant. To further investigate this, a DDR reporter was created by fusing the promoter of recA, a well-known DDR gene, with gfp. The reporter fusion was introduced, by integration at the amyE locus, into the wild type (LA174) and the pulcherrimin mutant (LA224), respectively. Pellicle biofilms developed in LBGM, and were collected every day over the course of 4 days and imaged using a fluorescence microscope. The wild-type reporter strain displayed increasing fluorescence over time (FIG. 4A), which was expected due to increasing accumulation of toxic molecules derived from metabolism that could damage DNA as the biofilm ages. Interestingly, the pulcherrimin mutant displayed much higher fluorescence compared with the wild type at each of the four time points that the samples were collected (FIG. 4B). The fluorescence of hundreds of individual cells from both the wild type and the pulcherrimin mutant collected at 48 h was quantified using MicrobeJ. FIG. 4C is the violin plot generated from the quantifications, where it shows that the values of fluorescence representing the activities of the DDR reporter P_{recA}-gfp are significantly higher in the pulcherrimin mutant (red) than in the wild type (blue). These results indicate that in the mature biofilm, cells of the pulcherrimin mutant likely undergo elevated stress that leads to more DNA damage compared with the wild type.

To further assess the implication of the above results, the wild-type and the pulcherrimin-mutant reporter strains were challenged with two different DNA-damaging agents. One of them was mitomycin C, an antibiotic known to cause DNA double stranded breaks and widely used to study the DDR in B. subtilis, and the other was hydrogen peroxide (H₂O₂), a well-known ROS that causes damage of several cellular structures including DNA. The assay consisted of growing cells under shaking conditions in LB broth for 2 hours followed by mitomycin C or H₂O₂ challenge for one hour at the final concentration of 0.25 μg/mL and 100 μM, respectively. Controls corresponded to untreated samples. After treatment, cells were collected, washed twice with PBS, and imaged under a fluorescence microscope. The P_{recA}-gfp reporter in the pulcherrimin mutant was found to display substantially higher fluorescence upon both DNA-damaging treatments compared with the wild type (FIG. 5A-D). These results suggest that lack of pulcherrimin rendered the mutant much more sensitive to external DNA-damaging agents than the wild type. It can be concluded that pulcherrimin plays a role in protecting cells by lowering DNA damage caused by both internal and external stresses.

To test whether levels of DNA damage are physically elevated in the pulcherrimin mutant, assays of spontaneous rifampicin resistance frequency were carried out as a measurement for DNA mutation rate and levels of DNA damage 58. To do so, both the wild type and the pulcherrimin mutant was grown for 24 h in LBGM supplemented with 0.2 mM FeCl₃. The cells were cultured in an iron-overloaded medium so they would be more prone to oxidative stress through the Fenton reaction and consequently more DNA damages if a protective mechanism is lacking. Cells of each strain were then plated onto LB agar supplemented with rifampicin (5 μg/mL) and incubated overnight. Spontaneous rifampicin-resistant mutants appeared on the plates of the wild-type strain as expected. Interestingly, on the plates of the pulcherrimin mutant, the number of the spontaneous rifampicin-resistant colonies appeared at a much higher rate than the wild type (FIG. 4D). This result supports the hypothesis that in the wild type, pulcherrimin protects cells by physically lowering DNA damage.

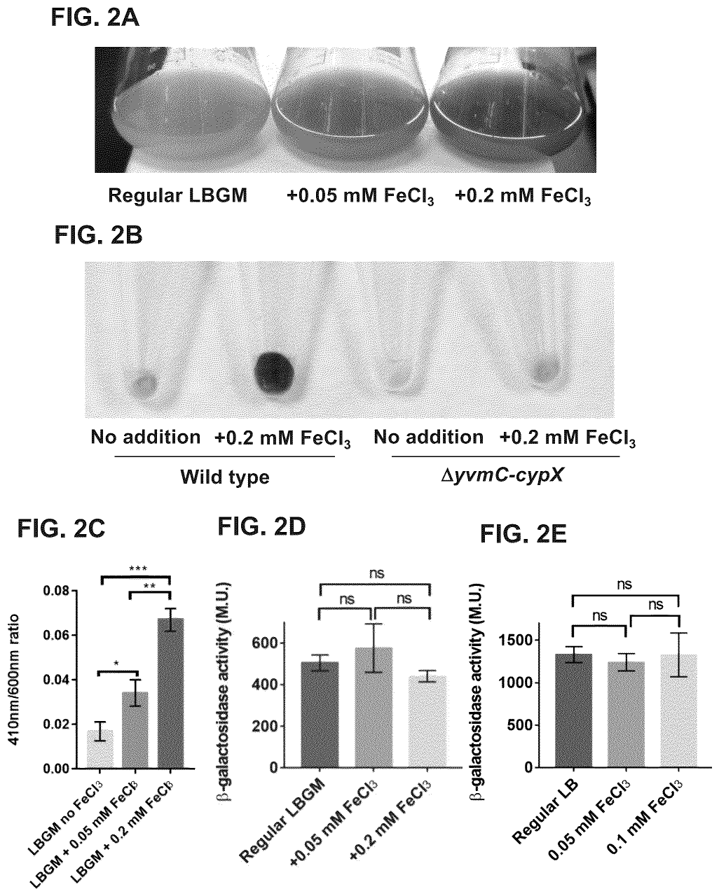

To further test if there are differences in cell survival under severe DNA damage stress between the wild type and the pulcherrimin mutant, cells were grown overnight in LBGM supplemented with 0.2 mM FeCl₃ (to ensure high pulcherrimin yield in the wild type; FIG. 2A). Each overnight culture was then split into two flasks. In one of the flasks, H₂O₂ was added at a final concentration of 10 mM for 10 min with shaking. The second flask, as a control, was not exposed to H₂O₂. Untreated and H₂O₂-treated cultures were both plated on LB agar plates and incubated overnight at 37° C. CFUs were counted the next day and the percent survival calculated for both strains. The average percent survival of the pulcherrimin mutant after exposure to H₂O₂ was approximately 20 times lower compared with the wild type (FIG. 5E). This result supports the idea that pulcherrimin protects cells and allows better survival of the cells under severe DNA damage stress.

### Pulcherrimin Production and DNA Damage Appear to be Anticorrelated in Cells in the Biofilm.

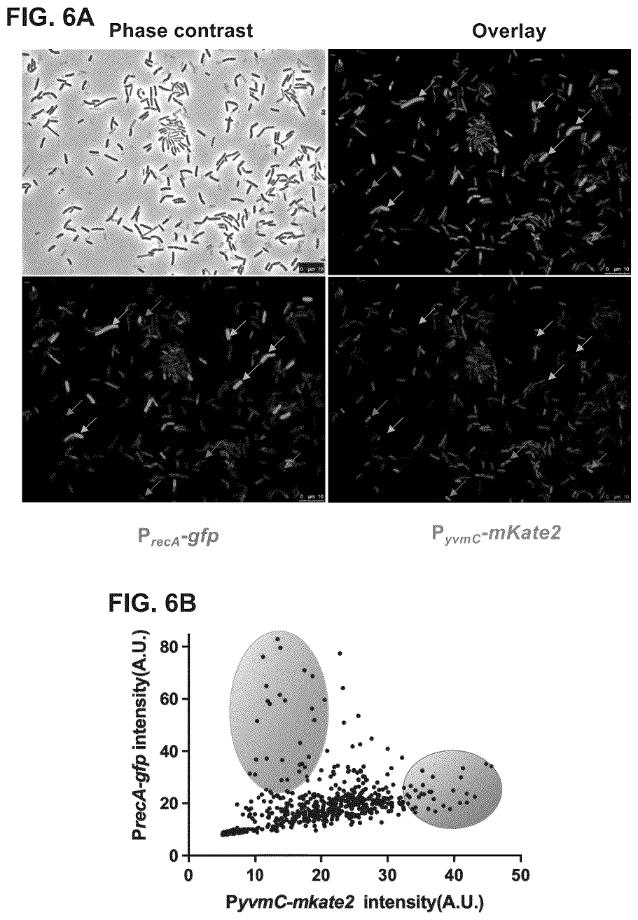

A dual reporter strain (LA233) carrying both the DDR reporter P_{recA}-gfp as a proxy for DNA damage, and the P_{yvmC}-mKate2 reporter for pulcherrimin production was constructed. This dual reporter strain was cultured in LBGM for pellicle development, harvested the pellicles after 48 hours (mature biofilm), mildly sonicated the pellicles to disrupt the bundles and chains, and imaged the cells under the fluorescence microscope applying the corresponding wavelengths for each reporter (FIG. 6A). The fluorescence was quantified for each reporter in individual cells and the results plotted in a dot plot graph where the DDR reporter's green fluorescence is displayed in the y axis, and the pulcherrimin reporter is on the x axis in fluorescent magenta (FIG. 6B). Each dot in the panel corresponds to a single cell bearing both reporters. The results depicted here show a significant amount of overlap among cells that displayed moderate fluorescence for both reporters, suggesting that a large proportion of cells in a mature biofilm express both reporters at intermediate levels. However, it is important to note that cells displaying high green fluorescence for the DDR reporter had very low magenta fluorescence for the pulcherrimin reporter (cells pointed by cyan-colored arrows in panels in FIG. 6A), and vice-versa (cells indicated by orange-colored arrows in the overlay image in FIG. 6A). Thus, it was observed that high pulcherrimin production anticorrelates with levels of DDR and probably of physical DNA damage. This, again, supports the hypothesis that pulcherrimin can protect cells from DNA damage.

### Pulcherrimin Lowers ROS Accumulation.

The present results suggest that pulcherrimin provides protection against DNA damage (FIGS. 3A-D and 4A-D). ROS levels in the wild type and the pulcherrimin mutant were measured utilizing a kit for total ROS measurements (Enzo Biosciences), which includes a cell-permeable and non-fluorescent dye that, once in contact with ROS (hydrogen peroxide or hydroxyl radicals), generates green fluorescence. Cells can then be imaged using a fluorescence microscope with a standard green filter (490/525 nm).

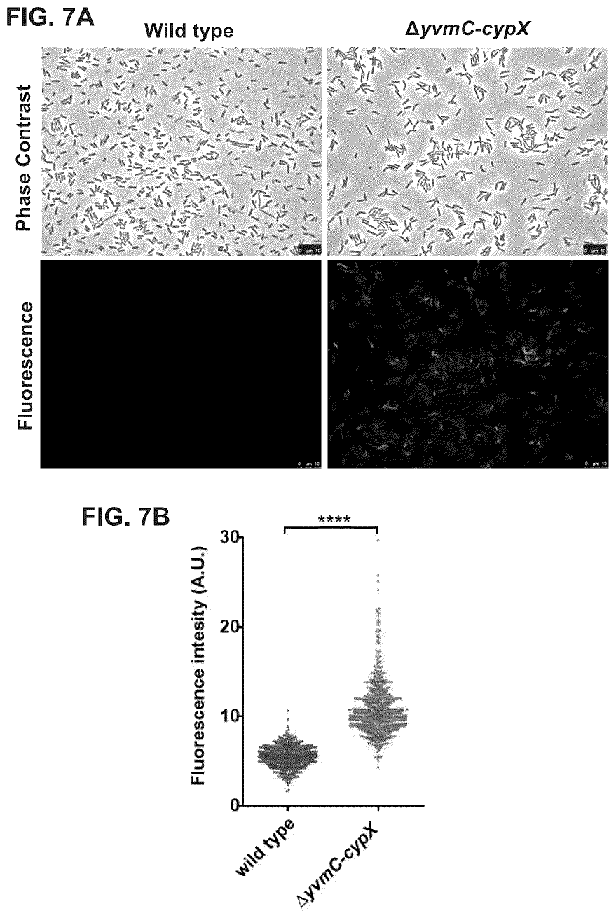

Cells of the wild type and the pulcherrimin mutant were grown overnight in LBGM supplemented with 0.2 mM FeCl₃ to enhance pulcherrimin yield. Cells were then incubated with the dye for 30 minutes in the dark, followed by three washes with PBS to remove excess dye, and then imaged. FIG. 7A corresponds to representative fluorescence microscopy images acquired for each sample, where the green fluorescence levels in the pulcherrimin mutant were clearly higher than in the wild type. Fluorescence of individual cells was also quantified using Image J and plotted in a violin plot, which exhibited a significant difference between the two strains (FIG. 7B). These results suggest that pulcherrimin is able to lower ROS accumulation and thus prevent damaging levels of oxidative stress to happen in the cells.

Pulcherrimin Production could Reduce Iron Toxicity by Lowering Iron Levels.

The results above provide both direct and indirect evidence that pulcherrimin reduces the oxidative stress in the cells and protects them from DNA damage.

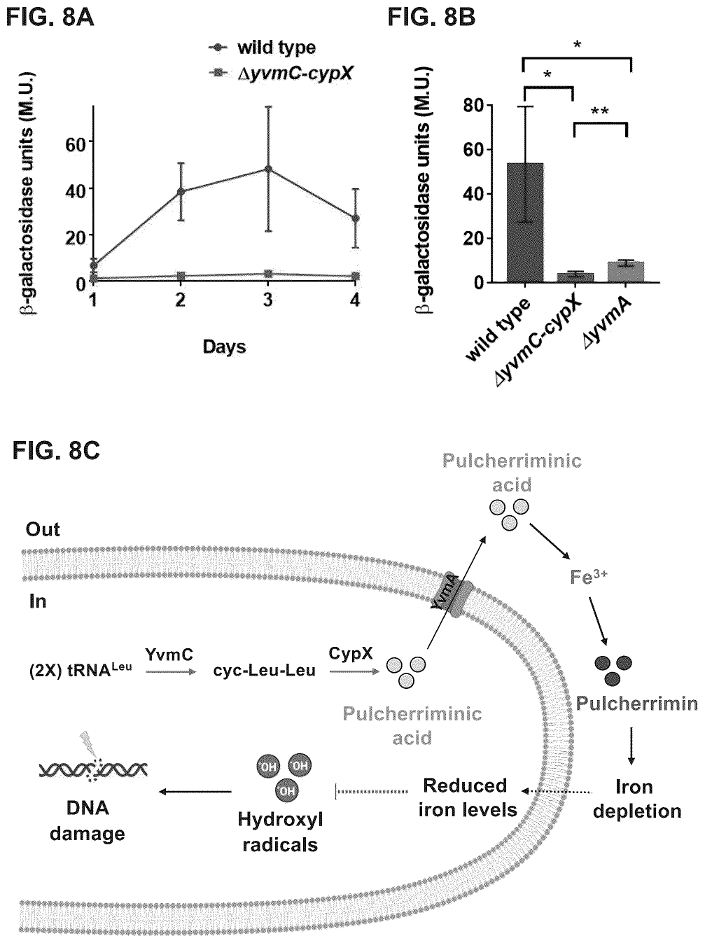

To test the hypothesis that by sequestering extracellular iron, pulcherrimin production can lower intracellular levels of iron, a transcriptional reporter with the promoter for the bacillibactin operon, whose expression highly anti-correlates with intracellular iron levels, fused to lacZ was constructed (P_{dhbA}-lacZ), and introduced into the wild type (YQ141) and the pulcherrimin mutant (LA148), respectively. Pellicle biofilms of the two reporter strains developed and were then harvested daily over the course of 4 days. β-Galactosidase assays were performed on collected samples and results shown in FIG. 8A. It is evident that the pulcherrimin mutant displayed much lower activities of the P_{dhbA}-lacZ reporter when compared with the wild type at every time point measured. This result supports the hypothesis that pulcherrimin production in the wild type significantly lowered intracellular iron levels and thus triggered upregulation of iron acquisition through bacillibactin and possibly other siderophores as well.

To further confirm that the observed difference in the P_{dhbA}-lacZ reporter activity between the wild type and the pulcherrimin mutant is caused by the formation of pulcherrimin extracellularly, and consequently iron depletion, the same reporter was introduced into the pulcherrimin transporter mutant, ΔyvmA (LA262). This yvmA gene mutation significantly reduces the secretion, but not biosynthesis, of pulcherriminic acid to the extracellular environment, as less pulcherrimin is observed in the media when compared with the wild type. Pellicle biofilms were similarly collected, and β-galactosidase assays performed to compare the activities of the P_{dhbA}-lacZ reporter in the wild type, the pulcherrimin biosynthetic mutant, and the pulcherrimin secretion mutant. The results show that both mutants (ΔyvmC-cypX and ΔyvmA) displayed a much lower promoter activation when compared with the wild type, with the pulcherrimin biosynthetic mutant (ΔyvmC-cypX) having the lowest promoter activation of all three (FIG. 8B). This confirms that the reason why the P_{dhbA}-lacZ reporter displays different activities between the wild type and the pulcherrimin mutant is indeed because of the formation of pulcherrimin extracellularly.

Together, the results described above further confirm that pulcherrimin production leads to extracellular iron depletion in wild-type biofilms. The iron depletion likely also significantly lowers intracellular iron levels because the promoter for the bacillibactin operon is strongly upregulated in the wild type compared with the pulcherrimin mutant, indicating an increased need for iron acquisition when pulcherrimin is present. To this end, several lines of evidence have been presented to support the working model on how pulcherrimin can function as an antioxidant to protect cells from oxidative stress and DNA damage during B. subtilis biofilm development (FIG. 8C). Pulcherriminic acid is produced inside the cell and is secreted to the extracellular environment by the dedicated transporter YvmA. If ferric iron ions are readily available, pulcherriminic acid molecules bind to them and turns into pulcherrimin. Because pulcherrimin is a largely insoluble molecule, it depletes the ferric iron ions when the precipitated complex accumulates in the extracellular environment. This iron depletion acts as a buffering mechanism that prevents too much iron from going into the cell. The cells are temporarily starving for iron when pulcherrimin is produced, which in this case is beneficial because the Fenton reaction is minimized and less hydroxyl radicals are generated intracellularly. Less hydroxyl radicals mean less DNA damage. Therefore, pulcherrimin, by lowering the ferric iron levels extracellularly, indirectly prevents cells from undergoing high levels of oxidative stress through the production of hydroxyl radicals.

### Example 2

Mitigating Candida albicans Virulence by Targeted Migration of Pulcherriminic Acid During Antagonistic Biofilm Formation by Bacillus subtilis

### Materials and Methods

Description of the microbial strains used in the study is summarized in Table 2. The strains were cultured and maintained in their appropriate selective media. B. subtilis, for example, was cultured, maintained, and tested in Lysogeny broth (BD Difco, US) (37° C., 150 rpm for 5 h) or LBGM medium (Shemesh and Chai, 2013). Whereas, C. albicans was grown in potato dextrose broth (PDB) (BD Difco, US), (37° C., 150 rpm, overnight) or PDB supplemented with agar, and/or Roswell Park Memorial Institute medium-1640 (RPMI) (Gibco, US).

TABLE 2

| Strain | Genotype |
|---|---|
| B. subtilis NCIB3610 | Wild-type strain |
| B. subtilis YC161 | amyE::Pₛₚₐₙₖ-gfp, chl^{R} in 3610 |
| B. subtilis DI103 | ΔepsHΔtasA in 3610 |
| B. subtilis RL3852 | ΔepsH in 3610 |
| B. subtilis SB505 | ΔtasA in 3610 |
| B. subtilis CY211 | ΔsinI in 3610 |
| B. subtilis RL4620 | Δspo0A in 3610 |
| B. subtilis NRS5532 | ΔcypX in 3610 |
| B. subtilis NRS5533 | ΔyvmC in 3610 |
| B. subtilis YC800 | ΔcypX ΔyvmC in 3610 |
| B. subtilis RL2662 | ΔsrfAA::erm in 3610 |
| B. subtilis LA20 | amyE::PyvmC-gfp, chl^{R} in 3610 |
| B. subtilis LA33 | ΔyvmC-cypX::tet^{R} amyE::Pₛₚₐₙₖ-yvmC-cypX, |
| | spec^{R} in 3610 |
| *B. subtilis LA43 | ΔyvmC-cypX::tet srfAA::erm amyE::Pspank- |
| | yvmC-cypX, spec^{R} in 3610 |
| C. albicans SC5314 | Clinical specimen - human |
| E. coli OP50 | Uracil auxotroph, feed for C. elegans |

### Single and Co-Culture Biofilm Model

To evaluate interactions within the dual-species consortium, an air-agar interface-based spatial proximity co-culture spot test was used. Roswell Park Memorial Institute Medium (RPMI)-1640, for example, was used tested for its ability to promote C. albicans hyphal filamentation. Physiological parameters that were best suited for both microbes were optimized, and the respective monocultures were continuously maintained for all the experiments.

### Biofilm Quantitative Analysis

The crystal violet biofilm quantification assays were carried out in 96-well microtiter plates (Tarsons Products Pvt. Ltd. India). Briefly, microbial cells were inoculated in an appropriate medium and incubated overnight at 37° C. without shaking. Cultures were then adjusted to OD600=0.01 and re-inoculated into fresh PDB medium, seeded on microtiter plates with or without surfactin (10, 25, 50 and 100 μg/mL) or purified pulcherrimin (0.05%, 0.1% and 0.2%), and incubated at 37° C. for 48 hours without shaking. Following incubation, the planktonic growth was measured at 600 nm, while the biofilms were quantified at 575 nm using a Biowave CO8000 cell density meter. The cells attached to the surface were then washed and stained for 20 min with 0.1% crystal violet, washed several times with sterile distilled water, and suspended in 95% ethanol. At 595 nm, plates were read and OD values were recorded.

### Microscopic Imaging

Biofilm inhibition assays were carried out in 96-well plates as described in Section 2.2. After 48 h of incubation, biofilms attached to polystyrene surfaces were washed several times, stained with SYTO® 9 dye and incubated in dark for 30 min. The excess dye was removed by washing and the plates were imaged using a GFP emission filter under a Nikon fluorescent microscope (Nikon Eclipse Ti2, Japan).

### Filamentation Assay

Yeast-hyphae (Y-H) switching assays were performed in liquid RPMI-1640 medium. C. albicans cultures (grown in PDB) were diluted to 1:100 in RPMI-1640 and incubated overnight at 37° C. (150 rpm) in the presence of different doses of either purified pulcherrimin (0.05%, 0.1% and 0.2%) or surfactin (10, 25, 50 and 100 μg/mL). Afterwards, 5 μL of the cultured suspensions were transferred to a glass microscopic slide and visualized with a Nikon fluorescent microscope (Nikon Eclipse Ti2, Japan).

### Assay for Colony Morphology

Yeast colonies were generated by spotting C. albicans (diluted to 1:100 in PDB) on PDA agar plates containing surfactin (10, 25, 50 and 100 μg/mL) and the non-treated (control) groups. The plates were incubated at 37° C. for 7 days and the hyphal prostration from colony edges was assessed using a phase-contrast mode of the Nikon fluorescent microscope (Nikon Eclipse Ti2, Japan).

### Nematode Survival Assay

Wild-type C. elegans were maintained on nematode growth medium (NGM) with E. coli OP50 as the feed. Synchronization was performed using known procedures. Briefly, C. elegans were collected by aspiration and bleached with 2% sodium hypochlorite and 0.5 N sodium hydroxide to get the eggs. Thereafter, the eggs were transferred to 48-well microliter plate and were incubated for 24 h at 22° C. for hatching. The hatched juveniles were transferred to fresh E. coli OP50 plates and incubated for 5-7 days to obtain adult nematode. Adults were subsequently used for toxicity, and bacterial or C. albicans colonization assays. For C. albicans infection of C. elegans, the adult nematodes, previously fed on E. coli OP50, were transferred to a C. albicans lawn on NGM agar plate for 4 h. After 4 h, the nematodes were collected in M9 buffer, pipetted into a 96-well plate and survival monitored for 7 days. The live and dead nematodes were counted under bright-field and DAPi filter and the nematode survival rates were estimated and plotted. The images of C. elegans were acquired using a Nikon fluorescent microscope (Nikon Eclipse Ti2, Japan).

### Statistical Analysis

All experiments were performed in quadruplicates and results are expressed as means±standard deviations. The student's t test was used to determine the significances of differences between treated and non-treated samples. Statistical significance was accepted for p values<0.05, and significant changes are indicated using asterisks in figures (*=p<0.05; **=p<0.01; ***=p<0.001).

### Results

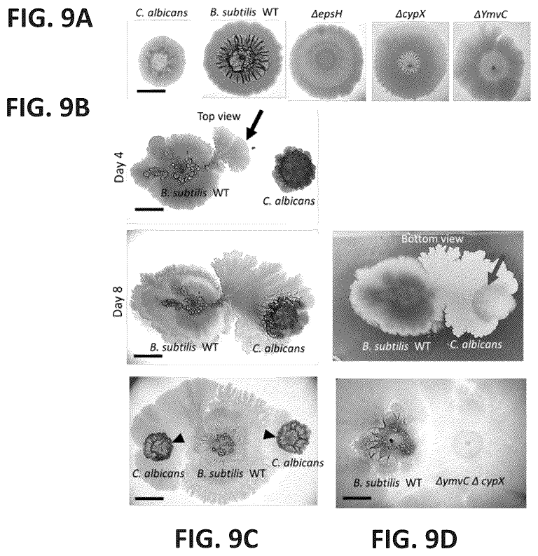

During activating biofilm formation pathway, B. subtilis produces colourless pulcherriminic acid (PA), synthesized intracellularly by two key enzymes, YvmC and CypX. In a two-step process, the enzymes convert two leucine tRNA molecules into pulcherriminic acid (PA), which is subsequently transported to the extracellular environment and binds ferric iron to form red-coloured pigment pulcherrimin. It was recently demonstrated that B. subtilis can produce pulcherrimin during biofilm formation in a legume-based media [Rajasekharan S K. et al., Microbial biotechnology. 2021; 14:1839-46; Amoah Y S, Nutrients. 2021; 13:4228]. A similar reddish-pink pigment production during formation of B. subtilis macrocolony biofilm onto PDA agar surface was observed (FIG. 9A). It was first confirmed that the produced pigment was pulcherrimin through characterizing phenotypically the yvmC and cypX mutant strains of B. subtilis. Furthermore, a reporter strain, bearing the P_{yvmC}-gfp transcriptional fusion, was used to monitor activation of the yvmC promoter within the B. subtilis macrocolony biofilm generated onto PDA surface.

Branching of B. subtilis Macrocolony Facilitates Pulcherriminic Acid Migration During Dual-Species Interactions

Probiotic Bacilli often antagonize pathogenic microbes during interspecies interactions The possible outcome of such interactions was investigated using a simplified in vitro model system in which selected species develop multicellular communities at the air-agar interface. Since B. subtilis forms macrocolony biofilm through secreting PA (FIG. 9A), the biochemical interplay between B. subtilis and C. albicans was tested during co-culture growth onto PDA surface. The spatial proximity test revealed intriguing branching phenotype of mature B. subtilis biofilm during dual-species interaction (FIG. 9B). It was subsequently found that the biofilm colony branch towards C. albicans macrocolony (FIG. 9B), leading to a targeted migration of PA from B. subtilis biofilm to the periphery of the C. albicans macrocolony (generated on either sides) (FIGS. 9B and 9C). The interplay between the wild-type and pulcherimin-deficient mutant cells was also investigated, demonstrating that pulcherimin is not migrated between different strains of the same species (FIG. 9D). During inter-species interactions, microbes sense and tend to react to neighboring organisms by bidirectional metabolite exchange. Thus, the results can be interpreted as a unidirectional pigment migration from B. subtilis biofilm colony towards community of pathogenic fungi.

Pulcherrimin Build-Up Results in Apparent Iron Deprivation in C. albicans

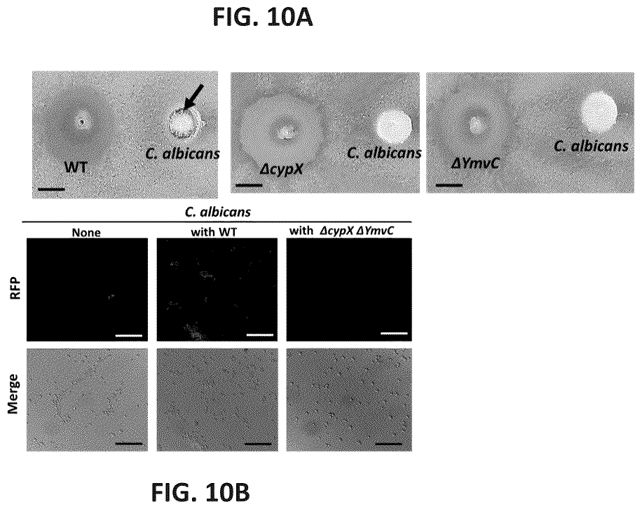

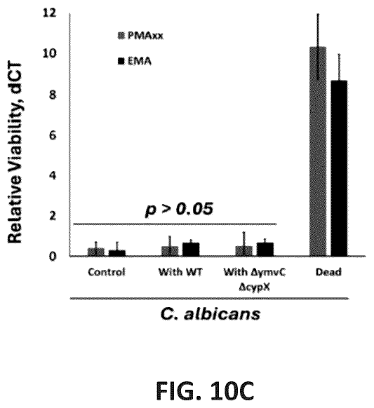

Similar to many pathogenic microorganisms, C. albicans utilizes intricate strategies to obtain iron from its environment. A substantial link between virulence and iron acquisition by the yeast pathogen is also evident. In the present model, pulcherrimin build-up was observed on C. albicans macrocolonies following the PA migration from B. subtilis biofilm (FIG. 9A). A build-up of pulcherrimin suggests an iron depletion within it, as the pulcherrimin formation apparently exhausts the iron in the medium surrounding C. albicans macrocolony (FIG. 10A). The Propidium iodide (PI) staining was used to assess the viability of C. albicans cells in the macrocolony, which revealed somewhat compromised cell viability in the area of pulcherrimin build-up (FIG. 10B).

To confirm this assumption, a qPCR based quantification method was used to show the cells viability in C. albicans macrocolony; however, no significant difference in viability of pathogenic cells using the cell viability qPCR assay was noted (FIG. 10C). It is likely that the pulcherrimin relay doesn't directly kill C. albicans cells.

B. subtilis Induces C. albicans Killing Through Hyphal Surface Colonization

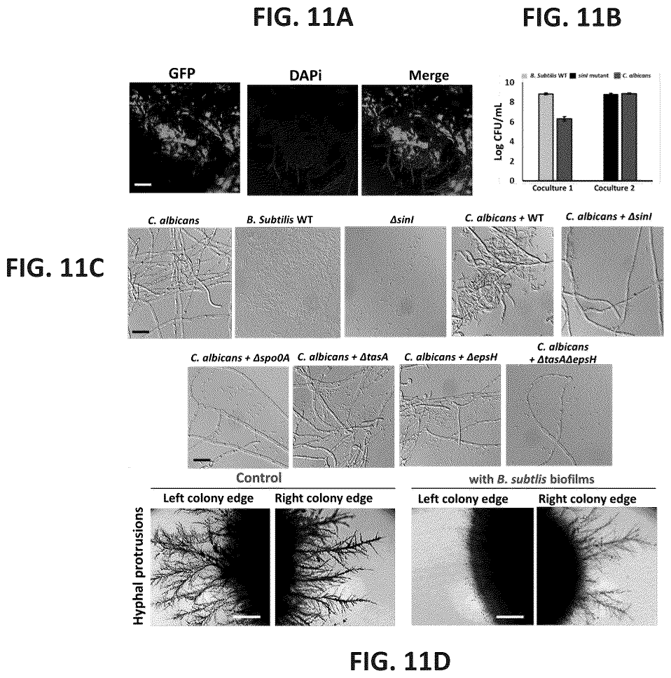

To further characterize the dual-species interactions, morphological and physiological changes during growth of the microbial cells (probiotic and pathogenic cells) in liquid co-culture was investigated. It was observed that the antagonistic activity in such co-culture conditions is dependent on B. subtilis' ability to colonize within the yeast community (FIGS. 11A and 11C). During optimizing the growth conditions of either microbe, it was found that the co-culture growth in RPMI-1640 media triggered robust hyphal filamentation in C. albicans; besides, the growth conditions was highly supportive for the biofilm bundles formation by B. subtilis (FIG. 11A). Thus, during co-culture growth in RPMI, B. subtilis demonstrated tremendous potential to colonize the hyphal filaments of C. albicans (FIGS. 11A and 11C).

Survivability of C. albicans was reduced by approximately two logs, when co-cultured with wild type cells, but not in case of the sinI mutant cells (which are unable to colonize the filaments). This result clearly indicate that hyphae colonization could be responsible for C. albicans killing during coculture growth (FIG. 11B). Similar hyphal colonization was observed in another biofilm promoting-LBGM medium, although C. albicans did not completely switch to filamental growth as noted in RPMI-1640. Besides massive hyphal colonization demonstrated by B. subtilis, it was further found that the hyphal protrusion from C. albicans macrocolonies were prevented during the coculture assays (FIG. 11D). Overall, it appears that B. subtilis is capable of halting and/or degrading hyphal filaments.

Using the Δspo0A, and matrix mutants (ΔtasA and ΔepsH), it was shows that B. subtilis colonization of yeast hyphal filaments is regulated by SpoA-SinI pathway (FIG. 11C). All of the biofilm mutants tested failed to adhere to C. albicans hyphal filaments, indicating that the SpoA-SinI pathway appears to be directly involved in regulating the observed phenomenon.

### A Defect in Surfactin Production Halts Pulcherrimin Migration and its Subsequent Antagonistic Activity

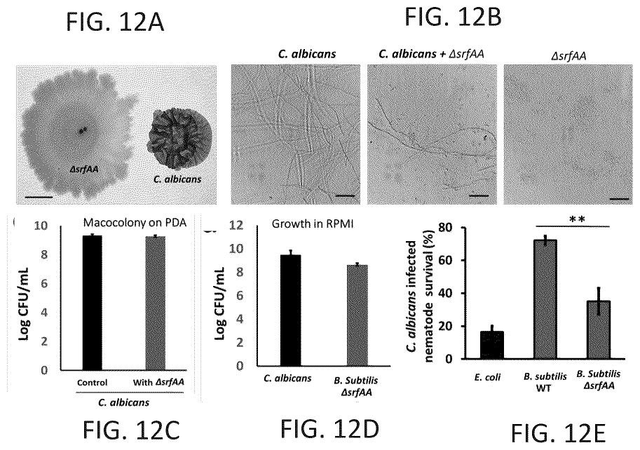

It was found that surfactin production mutant of B. subtilis (ΔsrfAA) failed to form branching phenotype, preventing them from reaching the neighbouring C. albicans macrocolony (FIG. 12A). Similarly, in liquid coculture assays, the ΔsrfAA mutant was unable to colonize hyphae (FIG. 12B) and had no effect on the viability of the yeast pathogen (FIGS. 12C and 12D). Also, in C. elegans model, B. subtilis WT demonstrated a remarkable ability to extend the lifespan of the C. albicans infected C. elegans, by reducing the C. albicans-induced killing of C. elegans. In contrast, the ΔsrfAA mutant could not prolong the lifespan of C. albicans-infected C. elegans (FIG. 12E), indicating that surfactin production by B. subtilis is instrumental in reducing C. albicans pathogenicity in C. elegans.

### Pulcherrimin Migration is Widespread During Inter-Species Bacterial Interactions

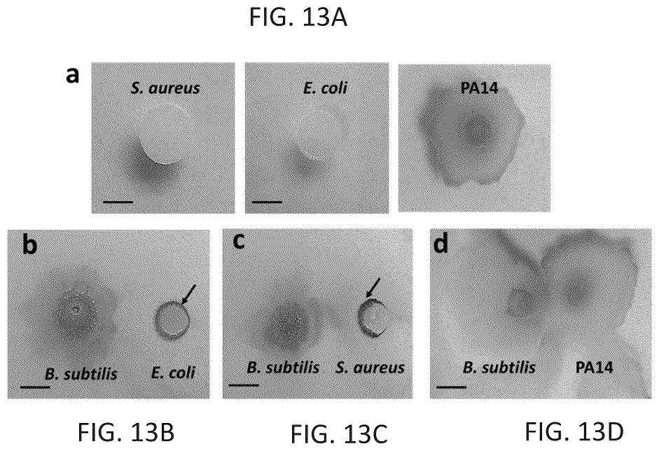

Interactions between B. subtilis cells and selected bacterial pathogens (E. coli, S. aureus, and P. aeruginosa) were studied using the co-culture model onto PDA interfaces (FIG. 13A). PA migration from B. subtilis macrocolony towards either E. coli or S. aureus colonies was comparable to that seen in case of C. albicans (FIGS. 13B and 13C), whereas, the ΔsrfAA mutant cells could not show any pulcherrimin build-up onto pathogenic macrocolony. Interestingly, the PA migration was not observed in case of P. aeruginosa PA14. Likely, the pathogen appeared to repel pulcherrimin build-up mediated antagonism by B. subtilis (FIG. 13D).

The Purified Bacillary Pulcherrimin Specifically Mitigates C. albicans Virulence

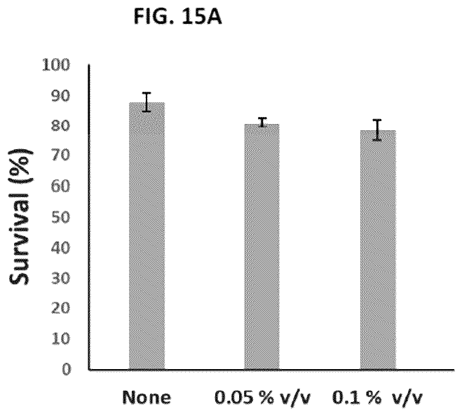

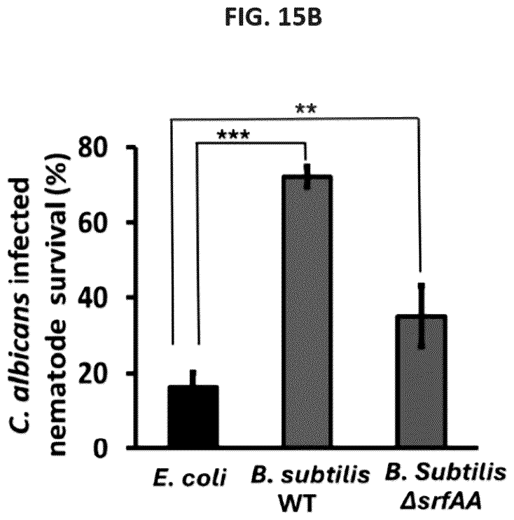

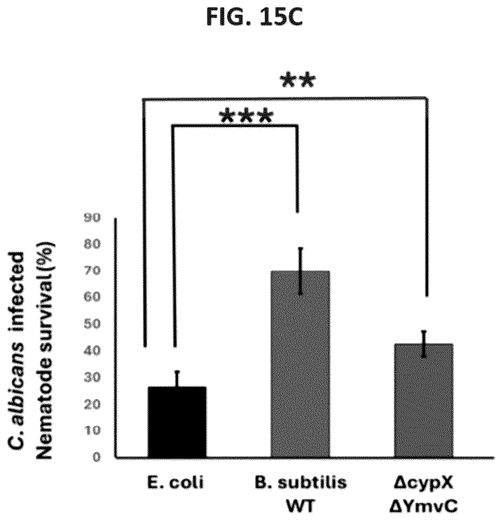

Pulcherrimin derived from B. subtilis appears to demonstrate remarkable antimicrobial properties. To test its anti-Candida activity, pulcherrimin was purified from B. subtilis biofilm colonies (herein designated as the pigment extract (PE)) and tested its activity against C. albicans growth, biofilm formation, and Y-H transitions. It appears that PE had no significant effect on the growth profile of C. albicans, however it notably inhibits C. albicans biofilm formation (FIG. 14A). Furthermore, PE was found to be effective in suppressing the Y-H switch (FIG. 14D). A strain was created that lacks surfactin production ability but can synthesize pulcherriminic acid when induced with Isopropyl β-D-1-thiogalactopyranoside (IPTG). Remarkably, the purified pulcherrimin from this strain (herein designated as the pigment extract 1 (PE1)) was able to suppress the biofilms and hyphae of C. albicans (FIGS. 14B-D). It was further demonstrated that the PE1 is not hazardous to an invertebrate model (FIG. 15A), supporting the safety of its possible antagonistic application.

In the C. elegans model, B. subtilis WT demonstrated a remarkable ability to extend the lifespan of the C. albicans infected C. elegans, by reducing the C. albicans-induced killing of C. elegans. In contrast, the ΔsrfAA mutant could not prolong the lifespan of C. albicans-infected C. elegans (FIG. 15B), indicating that surfactin production by B. subtilis is instrumental in reducing C. albicans pathogenicity in C. elegans. Pulcherrimin mutants were further shown to function in a similar manner like the surfactin mutant (FIG. 15C). It is likely that surfactin and pulcherrimin act separately to arrest C. albicans pathogenicity in nematodes.

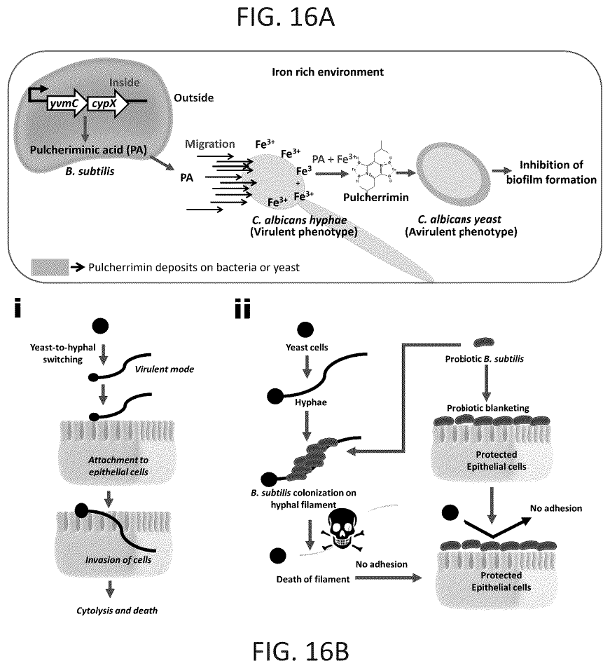

Microbial communities are constantly evolving in order to obtain iron, an essential element, from their surroundings. The present example demonstrates comprehensive antagonistic phenomena by which B. subtilis mitigates the virulence of C. albicans using coculture model systems. First, secretion of pulcherriminic acid by B. subtilis cells that subsequently forms pulcherrimin by complexing with ferric iron which accumulates on the C. albicans macrocolony, thus depleting the supply of free Fe³⁺ to the yeast population (FIG. 16A). Second, B. subtilis brutally colonizes hyphal filaments produced by C. albicans, creating a paradigm for suppressing C. albicans in humans (FIG. 16B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition of matter comprising genetically modified bacteria of the Bacillus genus which over-express cyclodipeptide synthase (YvmC) and/or cytochrome P450 oxidase (CypX), wherein said bacteria synthesize at least twice the amount of pulcherriminic acid as compared to the amount synthesized in a non-genetically modified bacteria of a corresponding strain.

2. The composition of matter of claim 1, wherein said genetically modified bacteria of the Bacillus genus comprise B. subtilis and/or B. licheniformis.

3. The composition of matter of claim 1, being essentially devoid of biofilm.

4. The composition of matter of claim 1, wherein said composition further comprises iron.

5. The composition of claim 4, wherein said amount of iron is such that the ratio of pulcherriminic acid:pulcherrimin is greater than 1:1.

6. The composition of matter of claim 1, further comprising a probiotic bacteria that is not capable of endogenously synthesizing pulcherriminic acid.

7. The composition of matter of claim 1, wherein said composition is a cell culture which comprises a culture medium.

8. A conditioned medium generated from the composition of matter of claim 7.

9. A method of generating pulcherriminic acid comprising culturing recombinant bacteria of the Bacillus genus genetically modified to over-express cyclodipeptide synthase (YvmC) and/or cytochrome P450 oxidase (CypX) in a culture medium under conditions that said bacteria synthesize at least twice the amount of pulcherriminic acid as compared to the amount synthesized in a non-genetically modified bacteria of a corresponding strain, thereby generating pulcherriminic acid.

10. The method of claim 9, wherein said conditions do not promote formation of a biofilm.

11. The method of claim 9, wherein said culture medium comprises starch fibers of a legume of a leguminous plant.

12. The method of claim 9, further comprising purifying said pulcherriminic acid from said culture medium following said culturing.

13. The method of claim 9, further comprising generating said genetically modified bacteria prior to said culturing.

14. A composition comprising:
(i) a probiotic bacteria; and
(ii) pulcherriminic acid and/or pulcherrimin,
wherein said probiotic bacteria in the composition is incapable of endogenously synthesizing pulcherriminic acid.

15. A method of treating a yeast infection in a subject in need thereof comprising administering to the subject a therapeutically effective amount of a purified preparation of pulcherriminic acid, thereby treating the yeast infection.

16. The method of claim 15, wherein said yeast infection is a Candida Albicans infection.

17. The method of claim 15, wherein said yeast is comprised in a biofilm.
